(19) United States
(12) Patent Application Publication
(10) Pub. No.: US 2010/0119530 A1
(43) Pub. Date: May 13, 2010

(54) Regulation of TLR Signaling by Complement

(76) Inventor: Wenchao Song, Bosemont, PA (US)

Correspondence Address: DRINKER BIDDLE & REATH;ATTN: INTELLECTUAL PROPERTY GROUP, ONE LOGAN SQUARE, SUITE 2000, PHILADELPHIA, PA, 19103-6996, US

(21) Appl. No.: 12/307,560

(22) PCT Filed: Jun. 29, 2007
(86) PCT No.: PCT/US07/15105

§ 371 Date: Oct. 30, 2009

(51) Int. Cl. A61K 39/38; A61P 35/00; A61P 29/00

(52) U.S. Cl. 424/184.1

(57) ABSTRACT

This invention provides methods of inducing the production of pro-inflammatory cytokines by activating Toll like Receptor (TLR) and the complement system. This invention further provides vaccines that contain compounds that activate a Toll like Receptor (TLR) and the complement system.

### CROSS REFERENCE TO RELATED APPLICATION

This application claims priority of U.S. Provisional Application Ser. No. 60/818,801, filed Jul. 6, 2006. This application is hereby incorporated in its entirety by reference herein.

### STATEMENT REGARDING FEDERALLY SPONSORED RESEARCH OR DEVELOPMENT

The invention described herein was supported in whole or in part by grants from The National Institutes of Health (Grant No. AI-63288, AI-49344, AI-44970, and GM-069736. The government has certain rights in the invention

### FIELD OF INVENTION

This invention provides: a method of inducing the production of pro-inflammatory cytokines in a subject by activating an anaphylatoxin receptor.

### BACKGROUND OF THE INVENTION

Toll is a Drosophila gene essential for ontogenesis and anti-microbial resistance. Several orthologues of Toll have been identified and cloned in vertebrates, namely Toll-like receptors (TLRs). Human TLRs are a growing family of molecules involved in innate immunity. TLRs are characterized structurally by a cytoplasmic Toll/interleukin-1 receptor (TIER) domain and by extracellular leucine-rich repeats. They are activated by pathogen-associated signature molecules such as LPS from Gram negative bacteria and components of yeast and mycobacteria. Most TLRs characterized so far activate the MyD88/interleukin-1 receptor associated kinase (IRAK) signalling pathway. Some TLRs (e.g. TLR3, TLR4) also activate MyD88-independent signaling pathways, resulting in the production of type I interferons and chemokines. Activation of TLRs leads to proinflammatory cytokine production, which may cause tissue injury. On the other hand, TLR signaling plays a crucial role in priming T cell immunity, which has relevance to vaccine development and anti-tumor immunotherapy, as well as to the treatment of autoimmunity.

The complement system is a biochemical cascade which helps clear pathogens from an organism. It is one part of the larger immune system. The complement system consists of a number of small proteins found in the blood, which work together to kill target cells by disrupting the target cell's plasma membrane. Over 20 proteins and protein fragments make up the complement system, including serum proteins, serosal proteins, and cell membrane receptors. These proteins are synthesized mainly in the liver, and they account for about 5% of the globulin fraction of blood serum. The complement system is not adaptable and does not change over the course of an individual's lifetime; as such it belongs to the innate immune system. However, it can be recruited and brought into action by the adaptive immune system.

Three biochemical pathways activate the complement system: the classical complement pathway, the alternative complement pathway, and the mannose-binding lectin pathway. The three pathways all generate homologous variants of the protease C3-convertase. The classical complement pathway typically requires antibodies for activation (specific immune response), while the alternate pathway can be activated by C3 hydrolysis or antigens without the presence of antibodies (non-specific immune response). Mannose-binding lectin pathway belongs to the non-specific immune response as well. C3-convertase cleaves and activates component C3, creating C3a and C3b and causing a cascade of further cleavage and activation events. C3b binds to the surface of pathogens leading to greater internalization by phagocytic cells by opsonization. C5a is an important chemotactic protein, helping recruit inflammatory cells. Both C3a and C5a have anaphylatoxin activity (mast cell degranulation, increased vascular permeability, smooth muscle contraction). C5b initiates the membrane attack pathway, which results in the membrane attack complex (MAC), consisting of C5b, C6, C7, C8, and polymeric C9. MAC is the cytolytic endproduct of the complement cascade; it forms a transmembrane channel, which causes osmotic lysis of the target cell. Kupffer cells and other macrophage cell types help clear complement-coated pathogens. As part of the innate immune system, elements of the complement cascade can be found in species earlier than vertebrates; most recently in the protostome horseshoe crab species, putting the origins of the system back further than was previously thought.

The complement system has the potential to be extremely damaging to host tissues meaning its activation must be tightly regulated. The complement system is regulated by complement control proteins, which are present at a high concentration in the blood plasma. Some complement control proteins are present on the membranes of self-cells preventing them from being targeted by complement. One example is CD59, which inhibits C9 polymerisation during the formation of the membrane attack complex.

It is thought that the complement system might play a role in many diseases with an immune component, such as Barraquer-Simons Syndrome, asthma, lupus erythematosus, glomerulonephritis, various forms of arthritis, autoimmune heart disease, multiple sclerosis, inflammatory bowel disease, and ischemia-reperfusion injuries. The complement system is also becoming increasingly implicated in diseases of the central nervous system such as Alzheimer's disease, and other neurodegenerative conditions. Deficiencies of the terminal pathway predispose to both autoimmune disease and infections (particularly meningitis, due to the role that the C56789 complex plays in attacking Gram negative bacteria). So far, the role of complement in these disease settings is thought to involve the direct effect of anaphylatoxins (C5a, C3a) and/or the membrane attack complex (C5b-9) per se as the end effectors.

### SUMMARY OF THE INVENTION

This invention provides, in one embodiment, a method of inducing the production of a pro-inflammatory cytokine in a subject, comprising the step of activating an anaphylatoxin receptor in a subject, thereby inducing the production of a pro-inflammatory cytokine in a subject.

In another embodiment, the present invention provides a method of preventing a Toll-like receptor (TLR) dependent inflammation in a subject, comprising the step of inhibiting an anaphylatoxin receptor in a subject, thereby preventing a Toll-like receptor (TLR) dependent inflammation in a subject.

In another embodiment, the present invention provides a method of inducing adaptive immune responses against an antigen in a subject, comprising the step of activating an anaphylatoxin receptor in a subject, thereby inducing adaptive immune responses against an antigen in a subject.

In another embodiment, the present invention provides a method of inhibiting adaptive immune responses against an antigen in a subject, comprising the step of inhibiting an anaphylatoxin receptor in a subject, thereby inhibiting adaptive immune responses against an antigen in a subject.

In another embodiment, the present invention provides a vaccine comprising an antigen, a Toll-like receptor (TLR) ligand, and an inducer of a complement system.

In another embodiment, the present invention provides a vaccine comprising an antigen, a Toll-like receptor (TLR) ligand, and an inhibitor of complement degradation.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1. LPS sensitivity of wild-type (WT) and DAF^{-/-} mice. ELISA assays of plasma levels of IL-6 (FIG. 1A), TNF-α (FIG. 1B) and IL-1β (FIG. 1C) in C57BL/6 WT and DAF^{-/-} mice at various time points after LPS challenge. FIG. 1D. Northern blot analysis of IL-6 mRNA levels in the spleen, lung and fat of C57BL/6 WT and DAF^{-/-} mice. Each lane represents an individual animal. FIG. 1E. ELISA assays of plasma IL-12p40 levels in C57BL/6 WT and DAF^{-/-} mice at various time points after LPS challenge. FIG. 1F. ELISA assays of plasma IL-6, TNF-α and IL-1β levels in Balb/c WT and DAF^{-/-} mice 3 hours after LPS challenge. G and H. Comparison of plasma IL-6, TNF-α (G) and IL-12p40, IL-12p70 (H) levels in C57BL/6 WT, DAF^{-/-} and CD59^{-/-} mice 3 hours after LPS challenge. I. Correlation plot of plasma IL-6 and LPS levels in C57BL/6 WT and DAF^{-/-} mice 3 hours after LPS challenge. N=4 for each group in FIGS. 1A-C and FIG. 1E. N=2 for each group in FIG. 1D. N=4-12 for each group in FIGS. 1F-I. Values shown are mean±SEM. *p<0.05, **P<0.01, Student t test.

FIG. 2. Effect of complement on LPS-induced cytokine production in vivo. FIG. 2A. ELISA assays of activated C3 products in plasmas of wild-type (WT) and DAF^{-/-} mice at various time points after LPS treatment. Percentage of C3 activation was relative to that of a mouse plasma sample activated in vitro by CVF. FIG. 2B. ELISA assays of plasma IL-6 and IL-12p40 levels in WT, DAF^{-/-}, C3^{-/-} and DAF^{-/-}/C3^{-/-} mice 3 hours after LPS challenge. FIG. 2C. ELISA assays of plasma IL-6 and IL-12p40 levels in WT mice 3 hours after CVF, LPS or CVF/LPS treatment. FIG. 2D. Effect of a C3a receptor antagonist (C3aRa) and a C5a receptor antagonist (C5aRa) on LPS-induced plasma IL-6 levels in DAF^{-/-} mice. Polyethylene glycol 400 (PEG) was used as a vehicle control. Antagonists were administered 30 minutes before LPS injection. FIG. 2E. ELISA assays of plasma IL-6 and IL-12p40 levels in WT, C3aR^{-/-} and C5aR^{-/-} mice 3 hours after LPS or LPS/CVF treatment. N=4-6 mice per group for FIGS. 2A-E. Values shown are mean±SEM. *p<0.05, **P<0.01, Student t-test.

FIG. 3. Effect of complement on LPS-induced cytokine production by splenocytes and peritoneal macrophages in vitro. FIG. 3A. ELISA assays of IL-6 production by wild-type (WT) and DAF^{-/-} mouse splenocytes in culture. Splenocytes from LPS-challenged (30 minutes before harvest) mice were cultured for 3 hours in the presence or absence of C5a (50 nM) and C3a (200 nM). FIG. 3B. and FIG. 3C. ELISA assays of IL-6 (FIG. 3B) and TNF-α (C) production by WT and DAF^{-/-} mouse peritoneal macrophages in culture. Cells were stimulated by various concentrations of LPS for 5 hours. FIG. 3D. ELISA assays of IL-6 production by WT and DAF^{-/-}/C3^{-/-} mouse peritoneal macrophages in culture. Cells were stimulated by 1000 ng/ml LPS for 5 hours. FIG. 3E. ELISA assays of IL-6 production by WT mouse peritoneal macrophages stimulated for 5 hours with LPS (100 ng/ml or 1000 ng/ml) in the presence or absence of C5a (50 nM) and C3a (200 nM). Cells from 4-5 mice were pooled and assayed in triplicate wells. Values shown are the mean±SEM. *p<0.05, **P<0.01, Student t test.

FIG. 4. Role of NF-κB activation and MAP kinase phosphorylation in the LPS sensitivity phenotype of DAF^{-/-} mice. FIG. 4A. Western blot analysis showing the time course of IκBα phosphorylation in wild-type (WT) and DAF^{-/-} mouse spleens after LPS challenge. Each time point represents an individual mouse. FIG. 4B. Western blot analysis of IκBα phosphorylation in the spleens of 4 WT and 4 DAF^{-/-} mice at 30 minutes after LPS challenge. C. Western blot analysis of IκBα levels in the spleens of 4 WT and 4 DAF^{-/-} mice at 60 minutes after LPS challenge. FIG. 4D. Effect of C5a (50 nM) on LPS (1000 ng/ml)-induced activation of an NF-kB luciferase reporter gene and TNF-α production in RAW264.7 cells. Cells were transiently transfected with the reporter gene plasmid together with a human C5aR cDNA construct. NT: no treatment. FIG. 4E Western blot analysis showing the time course of ERK1/2 phosphorylation in WT and DAF^{-/-} mouse spleens after LPS challenge. Each time point represents an individual mouse. FIG. 4F. Western blot analysis of JNK phosphorylation in the spleens of 4 WT and 4 DAF^{-/-} mice at 60 minutes after LPS challenge. Relative amount of each protein was expressed as the ratio between the protein and β-actin signals on Western blots.

FIG. 5 Complement regulates TLR2/6 and TLR9 activation. FIG. 5A. ELISA assays of plasma IL-6 levels in wild-type (WT) and DAF^{-/-} mice after zymosan treatment. FIG. 5B. ELISA assays of plasma IL-6 and IL-12p40 levels in WT and MyD88^{-/-} mice 3 hours after zymosan or zymosan/CVF treatment. FIG. 5C. ELISA assays of plasma IL-6 and IL-12p40 levels in WT, DAF^{-/-}, DAF^{-/-}/C3^{-/-} and DAF^{-/-}/C5R^{-/-} mice 3 hours after CpG treatment. FIG. 5D. ELISA assays of plasma IL-6 and IL-12p40 levels in WT mice 3 hours after CpG, CVF or CpG/CVF treatment. FIG. 5E. ELISA assays of plasma IL-12p40 levels in WT, C5aR^{-/-} and C3aR″ mice 3 hours after CpG or CpG/CVF treatment. N=2 mice for the MyD88^{-/-} groups in panel B, N=4-7 mice for all other groups. Values shown are the mean±SEM. *p<0.05, **p<0.001, Student t-test.

FIG. 6. Role of IL-10 in complement-mediated IL-12 inhibition. FIG. 6A. ELISA assays of plasma IL-10 levels in wild-type (WT) and DAF^{-/-} mice 3 hours after LPS, CVF or LPS/CVF treatment. FIG. 6B. ELISA assays of plasma IL-12p40 levels in WT and IL-10^{-/-} mice 3 hours after LPS or LPS/CVF treatment. FIG. 6C. ELISA assays of IL-10 production by cultured WT mouse peritoneal macrophages 5 hours after LPS and/or C5a (50 nM) and C3a (200 nM) stimulation. FIG. 6D. ELISA assays of IL-12p40 production by cultured WT mouse peritoneal macrophages 5 hours after LPS and/or C5a (50 nM) and C3a (200 nM) stimulation in the presence or absence of anti-IL-10 mAb (5 ng/ml). N=4-6 mice per group for FIG. 6A and FIG. 6B. Macrophages from 4-5 mice were pooled and assayed in triplicates in FIG. 6C and FIG. 6D. Values shown are the mean±SEM. *p<0.05, **p<0.001, Student t test.

FIG. 7. diagram showing proposed interaction between complement and the TLR pathways. PAMPs such as LPS and zymosan can activate both pathways. Activated complement regulates TLR signaling through the G protein-coupled anaphylatoxin receptors C5aR and C3aR, MAPKs, NF-kB and likely other transcription factors. In the absence of the complement regulatory protein DAF, complement activation and its effect on TLR signaling is amplified. The absence of DAF may be mimicked by strong complement activators such as CVF or pathological conditions such as sepsis.

FIG. 8. depicts bar graphs showing the combined effect of CVF (cobra venom factor, a complement activator) and LPS in the induction of serum IL-6 (FIG. 8A), serum TNF-α (FIG. 8B) and serum IL-1β (FIG. 8C). The effect of CVF on LPS-induced IL-12 production is shown in FIG. 8D. Sera analyzed were from mice that were non-treated wild-type (NT), CVF-treated wild-type (CVF), LPS-treated wild-type (LPS), LPS and CVF co-treated wild-type (LPS+CVF), LPS and CVF co-treated C3-deficient (C3ko), LPS and CVF co-treated C3a receptor-deficient (C3aRko) or LPS and CVF co-treated C5a receptor-deficient (C5aRko).

FIG. 9. depicts the effect of different sera characterized in FIG. 8 on Th-17 T cell differentiation. FACS analysis results of purified naive CD4 T cells from wild-type mice that were stimulated in vitro with plate-bound anti-CD3 and CD28 in the presence of specific cytokines or sera of control (naive mouse, corresponding to NT group in FIG. 8) or LPS-, CVF- or LPS+CVF-treated wild-type (non-specified) or different knockout (specified) mice. FIGS. 9A-C show CD4 T cells differentiation into Th-17 cells by IL-6 in the presence of TGF-β. FIG. 9D and FIG. 9E show the lack of effect of naive or CVF-treated mouse sera to drive Th-17 differentiation. FIG. 9F and FIG. 9G show the effect of serum from LPS-treated mice in driving Th-17 differentiation and the augmenting effect of CVF co-treatment. FIGS. 9H-J show the augmenting effect of CVF co-treatment on LPS-dependent Th-17 differentiation required C3 and C5a receptor but not C3a receptor.

FIG. 10. depicts a bar graph showing the synergistic effect of the complement activation product C5a and LPS in IL-6 production and its dependency on C5a receptor.

FIG. 11. depicts FACS analysis results of purified naive CD4 T cells from wild-type mice that were stimulated in vitro with plate-bound anti-CD3 and CD28 in the presence of specific cytokines or sera of control (naive mouse) or LPS-, C5a- or LPS+C5a-treated wild-type (WT) or C5a receptor (C5aR−/−) mice. FIGS. 11A-D show the capacity of CD4 T cells to differentiate into Th-17 cells by IL-6 in the presence of TGF-β□. Figure E and Figure F show the inability of naive mouse or C5a-treated mouse sera to drive Th-17 differentiation. Figure G and Figure H show the ability of serum from LPS-treated mice to induce Th-17 differentiation and the augmenting effect of C5a co-treatment of mice with LPS. FIGS. 11I-K show that the effect of C5a on LPS-dependent Th-17 differentiation required C5a receptor.

### DETAILED DESCRIPTION OF THE INVENTION

In one embodiment, the present invention provides a method of inducing the production of a pro-inflammatory cytokine in a subject, comprising the step of activating an anaphylatoxin receptor in a subject, thereby inducing the production of a pro-inflammatory cytokine in a subject. In another embodiment, the present invention provides a method of boosting the production of a pro-inflammatory cytokine in a subject, comprising the step of activating an anaphylatoxin receptor in a subject, thereby inducing the production of a pro-inflammatory cytokine in a subject. In another embodiment, the present invention provides a method of increasing the production of a pro-inflammatory cytokine in a subject, comprising the step of activating an anaphylatoxin receptor in a subject, thereby inducing the production of a pro-inflammatory cytokine in a subject. In another embodiment, the present invention provides that the terms “inducing”, “activating”, “increasing”, and “boosting” are used interchangeably.

In another embodiment, the present invention provides that activating an anaphylatoxin receptor in a subject results in at least 1 fold increase in the production of a pro-inflammatory cytokine. In another embodiment, the present invention provides that activating an anaphylatoxin receptor in a subject results in at least 2 folds increase in the production of a pro-inflammatory cytokine. In another embodiment, the present invention provides that activating an anaphylatoxin receptor in a subject results in at least 3 folds increase in the production of a pro-inflammatory cytokine. In another embodiment, the present invention provides that activating an anaphylatoxin receptor in a subject results in at least 4 folds increase in the production of a pro-inflammatory cytokine. In another embodiment, the present invention provides that activating an anaphylatoxin receptor in a subject results in at least 5 folds increase in the production of a pro-inflammatory cytokine. In another embodiment, the present invention provides that activating an anaphylatoxin receptor in a subject results in at least 8 folds increase in the production of a pro-inflammatory cytokine. In another embodiment, the present invention provides that activating an anaphylatoxin receptor in a subject results in at least 10 folds increase in the production of a pro-inflammatory cytokine. In another embodiment, the present invention provides that activating an anaphylatoxin receptor in a subject results in at least 15 folds increase in the production of a pro-inflammatory cytokine. In another embodiment, the present invention provides that activating an anaphylatoxin receptor in a subject results in at least 20 folds increase in the production of a pro-inflammatory cytokine, In another embodiment, the present invention provides that activating an anaphylatoxin receptor in a subject results in at least 25 folds increase in the production of a pro-inflammatory cytokine.

In another embodiment, the present invention provides that activating an anaphylatoxin receptor in a subject results in at least 30 folds increase in the production of a pro-inflammatory cytokine. In another embodiment, the present invention provides that activating an anaphylatoxin receptor in a subject results in at least 40 folds increase in the production of a pro-inflammatory cytokine. In another embodiment, the present invention provides that activating an anaphylatoxin receptor in a subject results in at least 50 folds increase in the production of a pro-inflammatory cytokine. In another embodiment, the present invention provides that activating an anaphylatoxin receptor in a subject results in at least 60 folds increase in the production of a pro-inflammatory cytokine. In another embodiment, the present invention provides that activating an anaphylatoxin receptor in a subject results in at least 70 folds increase in the production of a pro-inflammatory cytokine. In another embodiment, the present invention provides that activating an anaphylatoxin receptor in a subject results in at least 80 folds increase in the production of a pro-inflammatory cytokine. In another embodiment, the present invention provides that activating an anaphylatoxin receptor in a subject results in at least 90 folds increase in the production of a pro-inflammatory cytokine. In another embodiment, the present invention provides that activating an anaphylatoxin receptor in a subject results in at least 100 folds increase in the production of a pro-inflammatory cytokine.

In another embodiment, the present invention provides that cytokine production is measured by methods known to one of skill in the art.

In another embodiment, the present invention provides that the pro-inflammatory cytokine is IL-6. In another embodiment, the present invention provides that the pro-inflammatory cytokine is IL-10. In another embodiment, the present invention provides that the pro-inflammatory cytokine is TNFα. In another embodiment, the present invention provides that the pro-inflammatory cytokine is IL-1β. In another embodiment, the present invention provides that the pro-inflammatory cytokine is IL-8. In another embodiment, the present invention provides that the pro-inflammatory cytokine is LIF. In another embodiment, the present invention provides that the pro-inflammatory cytokine is GM-CSF. In another embodiment, the present invention provides that the pro-inflammatory cytokine is MIP-2. In another embodiment, the present invention provides a combination of pro-inflammatory cytokines.

In another embodiment, the present invention provides that the anaphylatoxin receptor is a C3a receptor (C3aR). In another embodiment, the present invention provides that the anaphylatoxin receptor is a C5a receptor (C5aR).

In another embodiment, the present invention provides that the C5aR is found on cells of the immune system. In another embodiment, the present invention provides that the C5aR is found on neutrophils. In another embodiment, the present invention provides that the C5aR is found on macrophages. In another embodiment, the present invention provides that the C5aR is found on mast cells. In another embodiment, the present invention provides that the C5aR is found on smooth muscle cells. The amino acid sequence of the receptor contains several N-terminal acidic residues, which may be involved in binding the basic C5a peptide.

In another embodiment, the present invention provides that the C3aR appears to be widely expressed in different lymphoid tissues. In another embodiment, the present invention provides that the C3a anaphylatoxin has a central role in inflammatory processes.

In another embodiment, the present invention provides that activating an anaphylatoxin receptor in a subject comprises activating the complement system in said subject. In another embodiment, the present invention provides that activating the complement system in a subject comprises administering to a subject an inducer of the complement system. In another embodiment, the present invention provides that activating the complement system in a subject comprises administering to a subject a composition comprising an inducer of the complement system. In another embodiment, the present invention provides that the inducer of the complement system is cobra venom factor (CVF). In another embodiment, the present invention provides that the inducer of the complement system is LPS. In another embodiment, the present invention provides that the inducer of the complement system is LOS (lipooligosacharride). In another embodiment, the present invention provides that the inducer of the complement system is zymosan. In another embodiment, the present invention provides that the inducer of the complement system is CpG. In another embodiment, the present invention provides that the inducer of the complement system is an immune complex. In another embodiment, the present invention provides that the inducer of the complement system is an apoptotic cell. In another embodiment, the present invention provides that the inducer of the complement system is a biomaterial surface such as cardio-pulmonary bypass tubing,.

In another embodiment, the present invention provides that activating the complement system in a subject comprises administering to a subject an inhibitor of complement degradation. In another embodiment, the present invention provides that the inhibitor of complement degradation is complement inhibitor decay-accelerating factor (DAF). In another embodiment, the present invention provides that DAF (CD55) is a 70 kDa membrane protein that regulates the complement system on the cell surface. In another embodiment, the present invention provides that DAF prevents the assembly of the C3bBb complex (the C3-convertase of the alternative pathway). In another embodiment, the present invention provides that DAF accelerates the disassembly of preformed convertase. In another embodiment, the present invention provides that DAF blocks the formation of the membrane attack complex. In another embodiment, the present invention provides that DAF is used as an adjuvant according to the methods of the present invention.

In another embodiment, the present invention provides that activating an anaphylatoxin receptor in a subject comprises administering to a subject a composition comprising a C3a protein, a C5a protein, or a combination thereof. In another embodiment, the present invention provides that C3a protein is used as an adjuvant according to the methods of the present invention. In another embodiment, the present invention provides that C5a protein is used as an adjuvant according to the methods of the present invention.

In another embodiment, the present invention provides that C3a protein is formed by C3-convertase that cleaves and activates component C3, creating C3a and C3b. In another embodiment, the present invention provides that C3a and C5a have anaphylatoxin activity. In another embodiment, an active fragment of C3a protein or C5a protein is used according to the methods of the present invention. In another embodiment, an analogue of C3a protein or C5a protein is used according to the methods of the present invention.

In another embodiment, the present invention provides that activating an anaphylatoxin receptor in a subject comprises administering to said subject a composition comprising an agonist of a C3a protein, a C5a protein, or a combination thereof. In another embodiment, the present invention provides that inducing the production of a pro-inflammatory cytokine in a subject comprises activating both a Toll-like receptor (TLR) and the complement system in a subject. In another embodiment, the present invention provides that activating the TLR in a subject comprises administering to a subject a composition comprising a lipopolysaccharide (LPS), or zymosan, or a combination thereof. In another embodiment, the present invention provides that activating the complement system in a subject comprises administering to a subject a composition comprising a lipopolysaccharide (LPS), or zymosan, or a combination thereof. In another embodiment, the present invention provides that activating both the TLR and the complement system in a subject comprises administering to a subject a composition comprising a lipopolysaccharide (LPS), zymosan, LOS, CpG, or a combination thereof.

In another embodiment, the present invention provides a method of preventing a TLR dependent inflammation in a subject, comprising the step of inhibiting an anaphylatoxin receptor in a subject, thereby preventing a TLR dependent inflammation in a subject. In another embodiment, the present invention provides a method of inhibiting a TLR dependent inflammation in a subject, comprising the step of inhibiting an anaphylatoxin receptor in a subject, thereby preventing a TLR dependent inflammation in a subject.

In another embodiment, the present invention provides that preventing a TLR dependent inflammation in a subject comprises inhibiting the production of a pro-inflammatory cytokine in a subject. In another embodiment, the present invention provides that inhibiting a TLR dependent inflammation in a subject comprises inhibiting the production of a pro-inflammatory cytokine in a subject. In another embodiment, the present invention provides that abrogating a TLR dependent inflammation in a subject comprises inhibiting the production of a pro-inflammatory cytokine in a subject.

In another embodiment, the present invention provides that the TLR is a TLR2. In another embodiment, the present invention provides that the TLR is a TLR3. In another embodiment, the present invention provides that the TLR is a TLR4. In another embodiment, the present invention provides that the TLR is a TLR5. In another embodiment, the present invention provides that the TLR is a TLR6. In another embodiment, the present invention provides that the TLR is a TLR7. In another embodiment, the present invention provides that the TLR is a TLR8. In another embodiment, the present invention provides that the TLR is a TLR9.

In another embodiment, the present invention provides ligands that activate the TLR of the invention. In another embodiment, the present invention provides that the ligand is a pathogen associated molecule. In another embodiment, the present invention provides that the ligand is a bacterial cell-surface component. In another embodiment, the present invention provides that the ligand is a bacterial cell-surface LPS. In another embodiment, the present invention provides that the ligand is a bacterial lipoproteins. In another embodiment, the present invention provides that the ligand is a bacterial lipopeptides. In another embodiment, the present invention provides that the ligand is a bacterial lipoarabinomannan.

In another embodiment, the present invention provides that the ligand is flagellin from bacterial flagella. In another embodiment, the present invention provides that the ligand is a double-stranded RNA of viruses. In another embodiment, the present invention provides that the ligand is an unmethylated CpG islands of bacterial DNA. In another embodiment, the present invention provides that the ligand is an unmethylated CpG islands of viral DNA.

In another embodiment, the present invention provides that the ligand is an endogenous ligand. In another embodiment, the present invention provides that TLRs function as dimers. In another embodiment, the present invention provides that TLRs function as homodimers. In another embodiment, the present invention provides that TLR2 forms heterodimers with TLR1. In another embodiment, the present invention provides that TLR2 forms heterodimers with TLR6. In another embodiment, the present invention provides that each dimer have different ligand specificity. In another embodiment, the present invention provides that TLR4's recognition of LPS, requires MD-2. In another embodiment, the present invention provides that LPS is administered with MD-2.

In another embodiment, the present invention provides that TLR ligands cause, in a complement-dependent manner, an elevated plasma concentration of pro-inflammatory cytokines. In another embodiment, the present invention provides that TLR ligands cause, in a complement-dependent manner, decrease in plasma IL-12 levels. In another embodiment, the present invention provides that TLR ligands and CVF, a potent complement activator, cause an elevated plasma concentration of pro-inflammatory cytokines.

In another embodiment, the present invention provides that the regulatory effect of complement on TLR-induced cytokine production is mediated by C5aR and C3aR. In another embodiment, the present invention provides that TLR ligands and CVF, induce mitogen-activated protein kinase and nuclear factor κB activation. In another embodiment, the present invention provides a strong interaction between complement and TLR signaling.

In another embodiment, the present invention provides that the C3aR and C5aR activate NF-kB. In another embodiment, the present invention provides that LPS activates the MAP kinases ERK1/2 and INK. In another embodiment, the present invention provides that MAPKs may be the key molecules linking TLR and complement system induction.

In another embodiment, the present invention provides that DAF regulates LPS-induced systemic complement activation. In another embodiment, the present invention provides that LPS incorporated into or associated with the cell membrane through micelle formation or binding to membrane proteins (e.g. CD14, TLR4).

In another embodiment, the present invention provides a novel mechanism by which complement promotes inflammation and modulates adaptive immunity and provides new insight into the interaction between two essential innate immune systems relevant to host-pathogen interaction. In another embodiment, the present invention provides a novel mechanism by which complement promotes inflammation and modulates adaptive immunity and provides new insight into the interaction between two essential innate immune systems relevant to autoimmunity. In another embodiment, the present invention provides a novel mechanism by which complement promotes inflammation and modulates adaptive immunity and provides new insight into the interaction between two essential innate immune systems relevant to the vaccine of the invention.

In another embodiment, the present invention provides that a TLR is expressed by an antigen-presenting cell.

In another embodiment, the present invention provides that the step of inhibiting an anaphylatoxin receptor in a subject comprises inhibition of the complement system in a subject. In another embodiment, the present invention provides that step of inhibiting an anaphylatoxin receptor in a subject comprises administering to a subject a C3aR antagonist, a C5aR antagonist, or a combination thereof. In another embodiment, the present invention provides that a C3aR antagonist is SB 290157. In another embodiment, the present invention provides that a C5aR antagonist is AcPhe. In another embodiment, the present invention provides that a C5aR antagonist is C5aRA A^{Δ71-73}.

In another embodiment, the present invention provides a method of inducing an immune response against an antigen in a subject, comprising the step of activating an anaphylatoxin receptor in a subject, thereby inducing an immune response against an antigen in a subject. In another embodiment, the present invention provides a method of activating the immune system against an antigen in a subject, comprising the step of activating an anaphylatoxin receptor in a subject. In another embodiment, the present invention provides that inducing an immune response against an antigen in a subject comprises the production of a pro-inflammatory cytokine in said subject. In another embodiment, the present invention provides that the step of activating an anaphylatoxin receptor in a subject comprises activating the complement system in a subject.

In another embodiment, the present invention provides a method of inhibiting an immune response against an antigen in a subject, comprising the step of inhibiting an anaphylatoxin receptor in a subject, thereby inhibiting an immune response against an antigen in a subject. In another embodiment, the present invention provides a method of abrogating an immune response against an antigen in a subject, comprising the step of inhibiting an anaphylatoxin receptor in a subject, thereby inhibiting an immune response against an antigen in a subject. In another embodiment, the present invention provides that inhibiting an immune response against an antigen in a subject comprises inhibiting the production of a pro-inflammatory cytokine in a subject. In another embodiment, the present invention provides that the step of inhibiting an anaphylatoxin receptor in a subject comprises inhibition of the complement system in a subject.

In another embodiment, the present invention provides a method of treating a Th17 cell mediated disease in a subject comprising the step of inhibiting complement system activation in a subject, thereby treating a Th17 cell mediated disease in a subject. In another embodiment, the present invention provides that Th17 is a CD4 effector T-cell subpopulation. In another embodiment, the present invention provides that the Th17T cells (a reference to their signature cytokine interleukin-17 (IL-17)), which is important in the pathogenesis of autoimmune diseases. In another embodiment, the present invention provides that Th17 differentiation is specified by a transcription factor that is also instrumental in lymphoid organogenesis.

In another embodiment, the present invention provides that a Th17 cell mediated disease is an autoimmune disease. In another embodiment, the present invention provides that the autoimmune disease is multiple sclerosis, lupus, inflammatory bowel disease, graft versus host disease, septic shock, arthritis, ischemia reperfusion injury, psoriasis, or transplant rejection. In another embodiment, the present invention provides that inhibiting complement system activation comprises administering to a subject a compstatin, anti-C5 monoclonal antibodies, anti-factor B monoclonal antibodies, anti-factor B monoclonal antibodies, anti-properdin monoclonal antibodies, recombinant extracellular domain of CRIg, recombinant DAF, recombinant MCP, recombinant DAF-MCP chimera protein, or any combination thereof. In another embodiment, the present invention provides that inhibiting complement system activation comprises administering to a subject a C5a antagonist such as C5aRA A8^{Δ71-73}.

In another embodiment, the present invention provides a vaccine comprising an antigen, a TLR ligand, and an inducer of the complement system. In another embodiment, the present invention provides that the antigen is a cancer antigen, bacterial antigen, or a viral antigen. In another embodiment, the present invention provides that the TLR ligand and the inducer of the complement system is a lipopolysaccharide (LPS), or zymosan. In another embodiment, the present invention provides that the inducer of the complement system is CVF. In another embodiment, the present invention provides that the inducer of the complement system is a C3a protein, a C5a protein or a combination thereof.

In another embodiment, the present invention provides a vaccine comprising an antigen, a Toll-like receptor (TLR) ligand, and an inhibitor of complement degradation. In another embodiment, the present invention provides that the antigen is a cancer antigen, bacterial antigen, or a viral antigen. In another embodiment, the present invention provides that the TLR ligand is a LPS or zymosan. In another embodiment, the present invention provides that the inhibitor of complement degradation is DAF.

In another embodiment, the present invention provides a vaccine comprising a nucleotide molecule and an adjuvant. In another embodiment, the present invention provides a vaccine comprising a protein used as an antigen and an adjuvant. In another embodiment, the present invention provides a vaccine comprising an organic molecule used as an antigen and an adjuvant. In another embodiment, the present invention provides a vaccine comprising an inorganic molecule used as an antigen and an adjuvant. In another embodiment, the adjuvant is a compound of the present invention. In another embodiment, the adjuvant is a LPS or zymosan. In another embodiment, the adjuvant a C3a protein, a C5a protein or a combination thereof. In another embodiment, the adjuvant a C3a protein agonist, a C5a protein agonist or a combination thereof. In another embodiment, the adjuvant is DAF. In another embodiment, the present invention provides a combined synergistic adjuvant comprising DAF and LPS. In another embodiment, the present invention provides a combined synergistic adjuvant comprising DAF and zymosan.

In other embodiments, the adjuvant of methods and compositions of the present invention further comprises Montanide ISA 51. Montanide ISA 51 contains a natural metabolizable oil and a refined emulsifier. In another embodiment, the adjuvant is GM-CSF. In another embodiment, the adjuvant is KLH. Recombinant GM-CSF is a human protein grown, in another embodiment, in a yeast (S. cerevisiae) vector. GM-CSF promotes clonal expansion and differentiation of hematopoietic progenitor cells, APC, and dendritic cells and T cells.

In another embodiment, the adjuvant further comprises a cytokine. In another embodiment, the adjuvant further comprises a growth factor. In another embodiment, the adjuvant further comprises a cell population. In another embodiment, the adjuvant further comprises QS21. In another embodiment, the adjuvant further comprises Freund's incomplete adjuvant. In another embodiment, the adjuvant further comprises aluminum phosphate. In another embodiment, the adjuvant further comprises aluminum hydroxide. In another embodiment, the adjuvant further comprises BCG. In another embodiment, the adjuvant further comprises alum. In another embodiment, the adjuvant further comprises an interleukin. In another embodiment, the adjuvant further comprises an unmethylated CpG oligonucleotide. In another embodiment, the adjuvant further comprises a quill glycosides. In another embodiment, the adjuvant further comprises a monophosphoryl lipid A. In another embodiment, the adjuvant further comprises a liposome.

In another embodiment, the adjuvant further comprises a bacterial mitogen. In another embodiment, the adjuvant further comprises a bacterial toxin. In another embodiment, the adjuvant further comprises a chemokine. In another embodiment, the adjuvant further comprises any other type of adjuvant known in the art. In another embodiment, the vaccine of methods and compositions of the present invention comprises 1 or more of the above adjuvants. In another embodiment, the vaccine comprises more than 2 of the above adjuvants. Each possibility represents a separate embodiment of the present invention.

In another embodiment, the vaccine is tested in human subjects, and efficacy is monitored using methods well known in the art, e.g. directly measuring CD4⁺ and CD8⁺ T cell responses, or measuring disease progression, e.g. by determining the number or size of tumor metastases, or monitoring disease symptoms (cough, chest pain, weight loss, etc). Methods for assessing the efficacy of a prostate cancer vaccine in human subjects are well known in the art, and are described, for example, in Uenaka A et al (T cell immunomonitoring and tumor responses in patients immunized with a complex of cholesterol-bearing hydrophobized pullulan (CHP) and NY-ESO-1 protein. Cancer Immun. Apr. 19, 2007; 7:9) and Thomas-Kaskel A K et al (Vaccination of advanced prostate cancer patients with PSCA and PSA peptide-loaded dendritic cells induces DTH responses that correlate with superior overall survival. Int j Cancer. Nov. 15, 2006; 119(10):2428-34). Each method represents a separate embodiment of the present invention.

In another embodiment, the present invention provides a method of overcoming an immune tolerance of a subject to an antigen, comprising administering to a subject an immunogenic composition comprising a compound of the present invention, thereby overcoming an immune tolerance of a subject.

“Tolerance” refers, in another embodiment, to a lack of responsiveness of the host to an antigen. In another embodiment, the term refers to a lack of detectable responsiveness of the host to an antigen. In another embodiment, the term refers to a lack of immunogenicity of an antigen in a host. In another embodiment, tolerance is measured by lack of responsiveness in an in vitro CTL assay. In another embodiment, tolerance is measured by lack of responsiveness in a delayed-type hypersensitivity assay. In another embodiment, tolerance is measured by lack of responsiveness in any other suitable assay known in the art. In another embodiment, tolerance is determined or measured as depicted in the Examples herein. Each possibility represents another embodiment of the present invention.

“Overcome” refers, in another embodiment, to a reversible of tolerance by a vaccine. In another embodiment, the term refers to conferment of detectable immune response by a vaccine. In another embodiment, overcoming of immune tolerance is determined or measured as depicted in the Examples herein. Each possibility represents another embodiment of the present invention.

In another embodiment, the present invention provides a vaccine for preventing cancer. In another embodiment, the present invention provides a vaccine for managing cancer. In another embodiment, the present invention provides a vaccine for treating cancer. In another embodiment, the present invention provides a vaccine for inhibiting cancer. In another embodiment, the present invention provides a vaccine for ameliorating cancer.

In another embodiment, the cancer is a melanoma. In another embodiment, the cancer is a sarcoma. In another embodiment, the cancer is a carcinoma. In another embodiment, the cancer is a lymphoma. In another embodiment, the cancer is a leukemia. In another embodiment, the cancer is mesothelioma. In another embodiment, the cancer is a glioma. In another embodiment, the cancer is a germ cell tumor. In another embodiment, the cancer is a choriocarcinoma. Each possibility represents a separate embodiment of the present invention.

In another embodiment, the cancer is pancreatic cancer. In another embodiment, the cancer is ovarian cancer. In another embodiment, the cancer is gastric cancer. In another embodiment, the cancer is a carcinomatous lesion of the pancreas. In another embodiment, the cancer is pulmonary adenocarcinoma. In another embodiment, the cancer is colorectal adenocarcinoma. In another embodiment, the cancer is pulmonary squamous adenocarcinoma. In another embodiment, the cancer is gastric adenocarcinoma. In another embodiment, the cancer is an ovarian surface epithelial neoplasm (e.g. a benign, proliferative or malignant variety thereof). In another embodiment, the cancer is an oral squamous cell carcinoma. In another embodiment, the cancer is non small-cell lung carcinoma. In another embodiment, the cancer is an endometrial carcinoma. In another embodiment, the cancer is a bladder cancer. In another embodiment, the cancer is a head and neck cancer. In another embodiment, the cancer is a prostate carcinoma.

In another embodiment, the cancer is an acute myelogenous leukemia (AML). In another embodiment, the cancer is a myelodysplastic syndrome (MDS). In another embodiment, the cancer is a non-small cell lung cancer (NSCLC). In another embodiment, the cancer is a Wilms' tumor. In another embodiment, the cancer is a leukemia. In another embodiment, the cancer is a lymphoma. In another embodiment, the cancer is a desmoplastic small round cell tumor. In another embodiment, the cancer is a mesothelioma (e.g. malignant mesothelioma). In another embodiment, the cancer is a gastric cancer. In another embodiment, the cancer is a colon cancer. In another embodiment, the cancer is a lung cancer. In another embodiment, the cancer is a germ cell tumor. In another embodiment, the cancer is an ovarian cancer. In another embodiment, the cancer is a uterine cancer. In another embodiment, the cancer is a thyroid cancer. In another embodiment, the cancer is a hepatocellular carcinoma. In another embodiment, the cancer is a thyroid cancer. In another embodiment, the cancer is a liver cancer. In another embodiment, the cancer is a renal cancer. In another embodiment, the cancer is a kaposis. In another embodiment, the cancer is a sarcoma. In another embodiment, the cancer is another carcinoma or sarcoma. Each possibility represents a separate embodiment of the present invention.

In another embodiment, the cancer is any other antigen-expressing cancer of the present invention known in the art. Each type of cancer represents a separate embodiment of the present invention.

In another embodiment, the present invention provides a vaccine for preventing infectious diseases. In another embodiment, the present invention provides a vaccine for managing infectious diseases. In another embodiment, the present invention provides a vaccine for treating infectious diseases. In another embodiment, the present invention provides a vaccine for inhibiting infectious diseases. In another embodiment, the present invention provides a vaccine for ameliorating infectious diseases.

In another embodiment, an antigen used by the methods of the present i s derived from or associated with the following organisms and/or diseases: Acanthamoeba, acquired immunodeficiency syndrome, adenovirus, Aedes albopictus, Aedes japonicus mosquito, African sleeping sickness, AHD, AIDS, alveolar hydatid disease, amebiasis, American trypanosomiasis, amnesic shellfish, Ancylostoma, Angiostrongylus, angiostrongyliasis, animal-borne diseases, Anisakis, anisakiasis, anthrax, antibiotic resistance, antimicrobial resistance, arboviral encephalitis, arboviral encephalitides, arenavirus infections, ascariasis, ascarids, Ascaris lumbricoides, aseptic (viral) meningitis, Asian mosquito, Aspergillus, aspergillosis, astrovirus infection, B. cepacia, Babesia, babesiosis, Bacillus anthracis, Bacterial and Mycotic Diseases, bacterial meningitis, balantidiasis, Balantidium, Bartonella henselae, Baylisascaris, Bayou virus, bilharzia, Black Creek Canal virus, Blastocystis hominis, blastomycosis, body lice, Bordetella pertussis, Borrelia burgdorferi, botulism, bovine spongiform encephalopathy, Brainerd diarrhea, broad (fish) tapeworm, Brucella, brucellosis, Brugia malayi infection, Brugia timori infection, BSE, Burkholderia cepacia, Burkholderia pseudomallei, calicivirus infection, Campylobacter, campylobacteriosis, Candida, candidiasis, Capillaria, capillariasis, Cat scratch disease, cat flea tapeworm infection, C. difficile, cercarial dermatitis, Cercopithecine herpesvirus, CFS, Chagas disease, chancroid, chickenpox, chikungunya fever, Chilomastix mesnili, Chlamydia, Chlamydia pneumoniae, Chlamydia psittaci, Chlamydia trachomatis, cholera, chronic fatigue syndrome, Chronic Wasting Disease (CWD), Ciguatera, CJD, CLM, Clonorchis, clonorchiasis, Clostridium difficile, Clostridium botulinum, Clostridium tetani, CMV, Coccidioides immitis, coccidioidomycosis, Corynebacterium diphtheriae, covert toxocariasis, Coxiella burnetti, Coxsackie A and B virus, crabs, Creutzfeldt-Jakob disease, Crimean-Congo hemorrhagic fever, cryptococcosis, Cryptococcus neoformans, cryptosporidiosis, Cryptosporidium, CSD, Culex mosquito, cutaneous larva migrans, CWD (Chronic Wasting Disease), Cyclospora infection, cyclosporiasis, cysticercosis, cytomegalovirus infection, or delusional parasitosis.

In another embodiment, an antigen used by the methods of the present is derived from or associated with the following organisms and/or diseases: dengue fever, dengue hemorrhagic fever, dengue hemorrhagic fever/dengue fever, dengue virus infection, diarrhea, diarrheagenic Escherichia coli, Dientamoeba fragilis infection, diphtheria, Diphyllobothrium infection, diphyllobothriasis, Dipylidium infection, disparities, dog flea tapeworm infection, dogs, dracunculiasis, drinking water safety, drug resistance Drug Service, CDC, ear infection, East African trypanosomiasis, Eastern equine encephalitis, Ebola hemorrhagic fever, Ebola virus infection, EBV, echinococcosis, echovirus infection, E. coli infection, Ehrlichia infection, ehrlichiosis, elephantiasis, emerging infectious diseases (listing, sites and publications about), encephalitis, encephalitis, arboviral, encephalitis, Eastern equine, encephalitis, Japanese, encephalitis, La Crosse, encephalitis, St. Louis, encephalitis, West Nile, Endolimax nana infection, Entamoeba coli infection, Entamoeba dispar infection, Entamoeba hartmanni infection, Entamoeba histolytica infection, Entamoeba polecki infection, enterobiasis, enterovirus infection (non-polio), epidemic typhus, Epstein-Barr virus, Erythema infectiosum, Escherichia coli infection, Fasciola infection, fascioliasis, fasciolopsiasis, Fasciolopsis buski infection, fever, scarlet, Fifth disease, filariasis, fish (broad) tapeworm infection, flu, or Francisella tularensis.

In another embodiment, an antigen used by the methods of the present is derived from or associated with the following organisms and/or diseases: Gambian sleeping sickness, GAS infection, gastroenteritis, viral, GBS infection , genital candidiasis, gerbils, German measles, Giardia infection, giardiasis, Global Migration and Quarantine, Division of, Gnathostoma infection, gnathostomiasis, gonorrhea, group A streptococcal infection, group B streptococcal infection, guinea pigs, Guinea worm disease, Haemophilus ducreyi infection, Haemophilus influenzae serotype b infection, hamsters, pet (diseases people can get from them), hand, foot, and mouth disease, hand hygiene in healthcare settings, Hansen's disease, hantavirus pulmonary, syndrome, head lice infestation, Helicobacter pylori infection, hematologic diseases, hemophilia, Hemorrhagic fever with renal syndrome, Hendra virus infection, hepatitis (viral), hepatitis A, hepatitis B, hepatitis C, hepatitis D, hepatitis E, Heterophyes infection, heterophyiasis, Hib disease, histamine fish poisoning, Histoplasma capsulatum infection, histoplasmosis, HIV infection, hookworm infection, HPIV, HPS, H. pylori infection, human ehrlichiosis, human immunodeficiency virus infection, human parainfluenzavirus infection, human parvovirus B19 infection, hymenolepiasis, Hymenolepis infection, iguanas, infectious mononucleosis, influenza, insects and their relatives (listing, disease information by type), intestinal roundworm infection, Iodamoeba buetschlii infection, Isospora infection,

In another embodiment, an antigen used by the methods of the present is derived from or associated with the following organisms and/or diseases: Japanese encephalitis, kala-azar, Kawasaki syndrome, Laboratory Network, Measles, La Crosse encephalitis, Lassa fever, LCMV, Legionella pneumophila infection, Legionnaires' disease, legionellosis, Leishmania infection, leishmaniasis, leprosy, Leptospira infection, leptospirosis, lice infestation, Listeria monocytogenes infection, listeriosis, Loa loa infection, Lockjaw, Lyme disease, lymphatic filariasis, lymphedema, lymphocytic choriomeningitis, MAC infection, mad cow disease, malaria, Marburg hemorrhagic fever, Marburg virus infection, marine toxins, measles, melioidosis, meningococcal disease, meningitis, Methicillin Resistant Staphylococcus aureus (MRSA), mice, Microsporidia infection, microsporidiosis, middle ear infection, Migration (Division of Global Migration and Quarantine), Migration, quarantine, and importation, molluscum contagiosum, monkeypox, mononucleosis, infectious mosquito-borne diseases, MRSA—Methicillin Resistant Staphylococcus aureus, mumps, murine typhus, Mycobacterium abscessus infection, Mycobacterium avium complex infection, Mycobacterium tuberculosis infection, or Mycoplasma pneumoniae infection.

In another embodiment, an antigen used by the methods of the present is derived from or associated with the following organisms and/or diseases: Naegleria infection, necrotizing fasciitis, Neisseria gonorrhoeae infection, neurocysticercosis, neurotoxic shellfish poisoning, new variant Creutzfeldt-Jakob disease, New York-1 virus infection, Nipah virus infection, Nocardia infection, nocardiosis, nonpathogenic intestinal amebae infection, non-polio enterovirus infection, Norovirus infection, Norwalk and Norwalk-like virus infection, nosocomial infections, nvCJD, ocular larva migrans, Onchocerca volvulus infection, onchocerciasis, OPC, opisthorchiasis, Opisthorchis infection, orf virus infection, oropharyngeal candidiasis, otitis media, paragonimiasis, Paragonimus infection, paralytic shellfish poisoning, parasitic roundworms, PCP infection, pediculosis, Pediculus infestation, Pediculus corporis infestation, Pediculus humanis capitis infestation, Pediculus pubis infestation, peptic ulcer disease, pertussis, PHN, pinworm infection, plague, Plasmodium infection, Pneumocystis carinii pneumonia: See Pneumocystis jiroveci pneumonia, Pneumocystis jiroveci pneumonia, pneumonia, polio, poliomyelitis, poliovirus infection, Pontiac fever, pork tapeworm infection, postherpetic neuralgia, Pseudomonas dermatitis, psittacosis, or pubic lice infestation.

In another embodiment, an antigen used by the methods of the present is derived from or associated with the following organisms and/or diseases: Q fever, rabies, rabies virus infection, raccoon roundworm infection, rat bite fever, rats, respiratory syncytial virus infection, rhinitis, Rickettsia rickettsii infection, Rickettsial diseases, Rift Valley fever, Rift Valley fever virus infection, ringworm, river blindness, RMSF, Rocky Mountain spotted fever, rotavirus, rotavirus infection, roundworm infection, intestinal, roundworm infection (parasitic), RSV infection, rubella, rubeola, runny nose, RVF infection, Salmonella infection, salmonellosis, Salmonella enteritidis infection, Salmonella typhi infection, Sarcoptes scabei infestation, SARS, scabies, scarlet fever, Schistosoma infection, schistosomiasis, Scientific Resources Program, scombrotoxic fish poisoning, scrub typhus, Severe acute respiratory syndrome, sexually transmitted diseases, sharps safety, shellfish (foodborne illnesses associated with), Shigella infection, shigellosis, shingles, Sin Nombre virus infection, slapped cheek disease, sleeping sickness, smallpox, sore mouth infection, Southern tick-associated rash illness, specimens (packing, importing/exporting, reference testing; through Scientific Resources Program), Spirillum minus infection, Sporothrix schenckii infection, sporotrichosis, Staphylococcus aureus infections, encephalitis, stomach flu, stomach ulcers, Streptobacillus moniliformis infection, Streptococcus infections, Streptococcus pneumoniae infection, streptococcal toxic shock syndrome, Strongyloides infection, strongyloidiasis, or syphilis.

In another embodiment, an antigen used by the methods of the present is derived from or associated with the following organisms and/or diseases: Taenia infection, taeniasis, Taenia solium infection, tapeworm (broad or fish) infection, tapeworm infection, TB, tetanus, three-day measles, thrush, tick-borne diseases (partial list), tick-borne relapsing fever, tick typhus, toxic shock syndrome, Toxocara canis, Toxocara cati, Toxocara infection, toxocariasis, Toxoplasma infection, toxoplasmosis, Treponema pallidum infection, Trichinella infection, trichinellosis, trichinosis, Trichomonas infection, trichomoniasis, trichuriasis, Trichuris infection, Trypanosoma brucei gambiense, Trypanosoma brucei rhodesiense, Trypanosoma cruzi infection, Trypanosoma infection, trypanosomiasis, TSS, tuberculosis, tularemia, typhoid fever, typhus fevers, ulcers, undulant fever, vaginal yeast infection, Vancomycin-intermediate/resistant Staphylococcus aureus, Vancomycin-resistant Enterococci, varicella, Varicella-Zoster virus infection, variola major, variola minor, VD, Vector-Borne Infectious Diseases, Division of venereal diseases, vesicular stomatitis with exanthema, VHF, Vibrio cholerae infection, Vibrio parahaemolyticus infection, Vibrio vulnificus infection, Viral and Rickettsial Diseases, viral (aseptic) meningitis, viral hemorrhagic fever, viral hepatitis, VISA, visceral larva migrans, von Willebrand disease, VRE, VRSA, vulvovaginal candidiasis, VZV infection, West African trypanosomiasis, Western equine encephalitis, West Nile viral encephalitis, West Nile virus infection, whipworm infection, whooping cough, Wuchereria bancrofti infection, yellow fever, yellow fever virus infection, Yersinia enterocolitica infection, Yersinia pestis infection, yersiniosis, zoonotic diseases, zoster.

### Experimental Detailes Section

### Materials and Methods

### Mice and Cell Line

C57BL/6-DAF^{-/-}, Balb/c-DAF^{-/-} and C57BL/6-CD59^{-/-} mice, deficient in the murine Daf-1 or CD59a gene, respectively, were generated by gene targeting and backcrossed. C57BL/6-TLR4^{-/-}, C57BL/6-IL-10^{-/-} and C57BL/6-C3^{-/-} (G6 backcross) mice were from The Jackson Laboratory (Bar Harbor, Me.). The C3^{-/-} mouse was further backcrossed in house to G11. C5aR^{-/-} and C3aR^{-/-} mice were generated by gene targeting as previously described and were backcrossed to G9 and G10, respectively, onto C57BL/6. C57BL/6-MyD88^{-/-} mice. C57BL/6-DAF^{-/-}C3^{-/-}, DAF^{-/-}C5aR^{-/-} and DAF^{-/-}TLR4^{-/-} mice were generated by crossbreeding the relevant single knockout strains. Gender- and age-matched wild-type (WT) mice were purchased from The Jackson Laboratory. Mice were housed in a specific pathogen-free facility and all experimental protocols were approved by the Institutional Animal Care and Use Committee.

The RAW264.7 murine macrophage cell line was obtained from American Type Culture Collection (Manassas, Va.) and maintained in DMEM (Invitrogen, N.Y.) with 10% FCS (Hyclone, Utah).

### Reagents

Ultrapure LPS (E. coli K12) was obtained from InvivoGen (San Diego, Calif.). In some experiments, LPS (E. Coli. 026:B6, Pheno/water extracted) from Sigma-Aldrich (St. Louis, Mo.) was used. These LPS produced similar results when tested in our experiments. Zymosan A derived from Saccharomyces cerevisiae, recombinant human C5a, anti-mouse β-actin monoclonal antibody, horse radish peroxidase (HRP)-conjugated rabbit anti-mouse IgG were from Sigma-Aldrich (St. Louis, Mo.). Zymosan was boiled in saline for 90 minutes and then centrifuged for 30 minutes at 4000 rpm, resuspended in saline at 50 mg/ml and stored at −20° C. CpG 1826 (5′-TCCATGACGTFCCTGACGTT-3′) was synthesized by Oligos Etc. (Wilsonville, Oreg.). C3a receptor antagonist (SB290157) was from Calbiochem Inc. (La Jolla, Calif.) and was prepared by dissolving in 20% PEG400 (USB Corporation, Cleveland, Ohio) in saline just before use. Cobra venom factor (CVF) was from Quidel Corporation (San Diego, Calif.). Recombinant human C3a was from Complement Research Technologies (San Diego, Calif.). FITC-conjugated anti-F4/80 and ELISA kits for mouse IL-6, IL-12p40, IL-12p70, IL-1β and IL-10 were from BD Pharmingen (San Diego, Calif.). Rabbit anti-mouse p-ERK, p-JNK, p-IκB and IκB were from Cell Signaling Technology (Beverly, Mass.). Goat anti-rabbit IgG-HRP was from BioRad Laboratories (Hercules, Calif.). Anti-mouse IL-10 mAb (clone JES052A5) and ELISA kit for mouse TNF-α was from R&D System (Minneapolis, Minn.). Thioglycollate Medium (Brewer Modified) was from Becton Dickinson Microbiology System (Sparks, Md.).

Treatment of Mice with TLR Ligands

Mice were injected with the following TLR ligands: LPS (20 mg/kg in PBS, i.p.), zymosan (1 g/kg in 0.9% saline, i.p.), CpG (20 mg/kg in PBS, i.p.). In some experiments, mice were also treated with CVF (15 U/mouse in saline, i.p.), SB290157 (30 mg/kg in 20% polyethylene glycol 400 in saline, i.p.), AcPhe (50 μg/mouse in PBS, i.p.). EDTA (20 mM) anti-coagulated blood samples were collected from the tail vein or vena cava. Plasma was prepared by centrifugation at 1000×g for 15 min at 4° C. and stored as small aliquots at −80° C.

### Cytokine Assays

IL-6, TNF-α, IL-12p40, IL-12p70, IL-1β, and IL-10 levels were determined using ELISA kits. Detection range was 15.6˜1000 pg/ml for IL-6 and IL-12p40, 23.4˜1500 pg/ml for TNF-α, 62.5-4000 pg/ml for IL-12p70, 31.3˜2000 pg/ml for IL-1β and IL-10.

### Complement Activation Assays

Levels of C3 activation fragments (C3b/iC3b/C3c) in plasma were measured by a sandwich ELISA. For quantification purposes, 1/500 serial dilutions of CVF-activated WT mouse plasma (prepared by adding 2.5 μg (1.2 units) of CVF to 50 μl plasma and incubating for 1 h at 37° C.) were used as a reference, and complement activation in all testing samples was normalized to this reference sample.

### Harvest and Culture of Mouse Splenocytes and Peritoneal Macrophages

Spleens were harvested 30 minutes after LPS injection and single splenocytes were prepared as described²³. Cells were cultured at 7.5×10⁶ cells/well in 0.2 ml DMEM complete medium (10% FBS, 2 mM L-glutamine, 10 mM Hepes, 0.1 mM nonessential amino acids, 100 U penicillin-streptomycin, 50 mM 2-mercaptoethanol, and 1 mM sodium pyruvate) with or without C3a (200 nM) and C5a (50 nM).

To prepare peritoneal macrophages, mice were injected with 2 ml of sterile 3% thioglycolate broth (i.p.). After 4 days, elicited cells were harvested by peritoneal lavage with cold Ca²/Mg²⁺-free PBS. Cells (1×10⁶/well) were seeded into 6-well plates and cultured in RPMI1640 (GIBCO, Grand Island, N.Y.) supplemented with 10% FBS, 50 μM 2-mercaptoethanol, and 1% penicillin-streptomycin with 5% CO₂. After 2 hours, non-adherent cells were removed by pipetting and gentle washing_{—} The remaining cells, confirmed to be mainly (>90%) macrophages by F4/80 staining, were cultured and stimulated with LPS (0.1 ng to 1 μg/ml) in the presence or absence of C5a (50 nM) and C3a (200 nM).

In some experiments, anti-IL-10 mAb was also added to the cell culture at 5 ng/ml. All cultures were analyzed for cell viability by the metabolic MIT assay as described previously.

### Northern Blot and Western Blot Analyses

Tissue RNAs were prepared using the TRIzol reagents (Life Technologies, Invitrogen) and Northern blot was performed as described previously¹⁶. IL-6 cDNA probe was synthesized by RT-PCR using 5′-GAGTTGTGCAATG GCAATTC-3′ and 5′-GTGTCCCAACA TTCATATTG-3′ as primers. Signals from Western blot were visualized by the ECL (Enhanced Chemiluminescence) System (Amersham Bioscieces) and detected by the FUJI ImageReader. Signal intensity was quantified using MultiGauge V3.0 and level of the protein of interest was expressed as the ratio of the specific signal over that of β-actin.

### Measurement of Plasma LPS Levels

Plasma LPS levels were determined using the Pyrochrome Kit from Associates of CAPE COD Incorporated (East Falmouth, Mass.). Plasma samples were treated at 70° C. for 10 minutes before assays to heat-inactivate serine proteases. Levels were expressed as units/ml of endotoxin.

### Transfection of RAW 264.7 Cells and Luciferase Reporter Gene Assay

RAW 264.7 cells were co-transfected with NF-kB Luc (Clontech, Palo Alto, Calif.), human C5aR in pcDNA3 (UMA cDNA resource center, Rolla, Mo.) and the Rellina control vector (Promega, Madison, Wis.) using the Amaxa Nucleofector apparatus (Amaxa Biosystems, Germany). The RAW 264.7 cells expressed detectible levels of endogenous C5aR as assessed by FACS and RT-PCR. After transfection, they also became weakly positive for human C5aR as assessed by FACS. Twenty-four hours after transfection, cells were stimulated with LPS (100 ng/ml) and/or C5a (100 nM) for 5 hours and luciferase activity was measured by using the Dual-Luciferase Reporter Assay system (Promega, Madison, Wis.) and a luminometer (Tuner Biosystems). Cell culture supernatants were collected for IL-6 and TNF-α assays by ELISA. All assays were performed in triplicates.

### EXAMPLE 1

### DAF^{-/-} Mice were Hyper Responsive to LPS Challenge

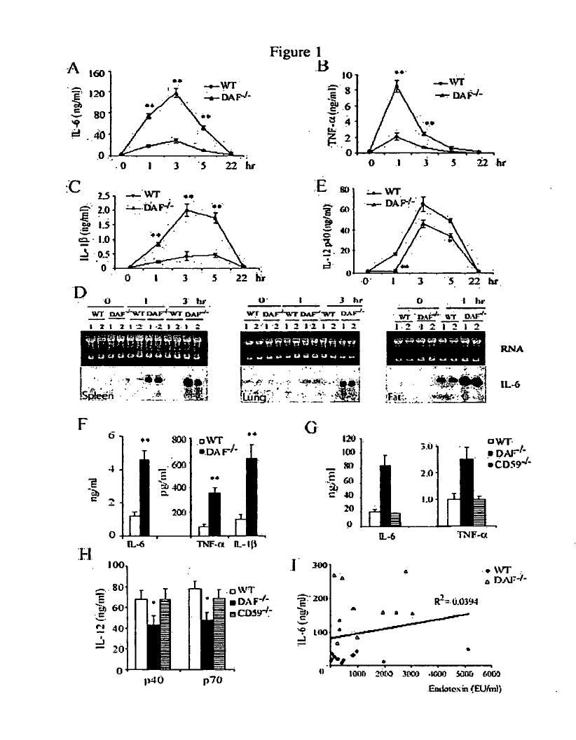

DAF is a LPS-binding protein, thus, the initial objective was to determine if DAF might play a role in LPS signaling in vivo. To achieve this goal, C57BL/6 wild-type and DAF^{-/-} mice were challenged with a sub-lethal dose of LPS (20 mg/kg). DAF^{-/-} mice developed more severe symptoms of endotoxin shock than wild-type mice (lack of activity, raised fur and hunched back posture). Consistent with this observation, plasma concentrations of IL-6, TNF-α and IL-1β were strikingly elevated (P<0.001) in DAF^{-/-} mice than in wild-type mice at 1 and 3 hours after LPS challenge (FIG. 1A-1C). Plasma IL-6 and IL-1□ levels remained significantly (P<0.001) elevated at 5 hr in the mutant mice but all three cytokines returned to baseline levels by 22 hours in both groups of mice (FIG. 1A-1C). By Northern blot analysis, we also detected markedly elevated IL-6 mRNA levels in the spleen, lung and adipose tissues of DAF^{-/-} mice at 1 or 3 hr (FIG. 1D). Conversely, we found that plasma IL-12p40 concentration was lower in DAF^{-/-} mice than in wild-type mice (FIG. 1E).

Similar increases in plasma IL-6, TNF-α and IL-1β concentrations 3 hr after LPS challenge were observed in Balb/c DAF^{-/-} mice (FIG. 1F), demonstrating that LPS hypersensitivity in DAF^{-/-} mice was independent of the genetic background. To determine whether the phenotype was related to the absence of DAF as a GPI-anchored protein from the cell surface, the LPS response of mice deficient in CD59 was studied, another GPI-anchored membrane complement regulatory protein that inhibits the terminal step of complement activation³¹. It was found that, unlike DAF^{-/-} mice, CD59^{-/-} mice secreted normal amounts of IL-6, TNF-α, IL-12p40 and IL-12p70 (FIG. 1G, 1H). These data indicated that the regulatory role of DAF in LPS signaling in vivo was specific.

Because human DAF has been shown to be a LPS-binding protein, the hypothesis that cellular DAF may serve as a ‘LPS sink’ was examined so that in its absence a higher effective plasma LPS concentration was achieved in DAF^{-/-} mice after LPS injection, potentially accounting for the observed phenotype in these mice. Plasma LPS concentrations were measured in wild-type and DAF^{-/-} mice 3 hours after LPS injection, but did not find significant differences between the two groups of mice (925±413 and 1200±307 EU/ml for wild-type and DAF^{-/-}, respectively. n=12, p=0.599, Mann-Whitney Test), nor did any correlation between plasma LPS and IL-6 concentrations was found in either wild-type or DAF^{-/-} mice (FIG. 11).

### EXAMPLE 2

### Increased Complement Activation was Responsible for the Altered LPS Response in DAF^{-/-} Mice

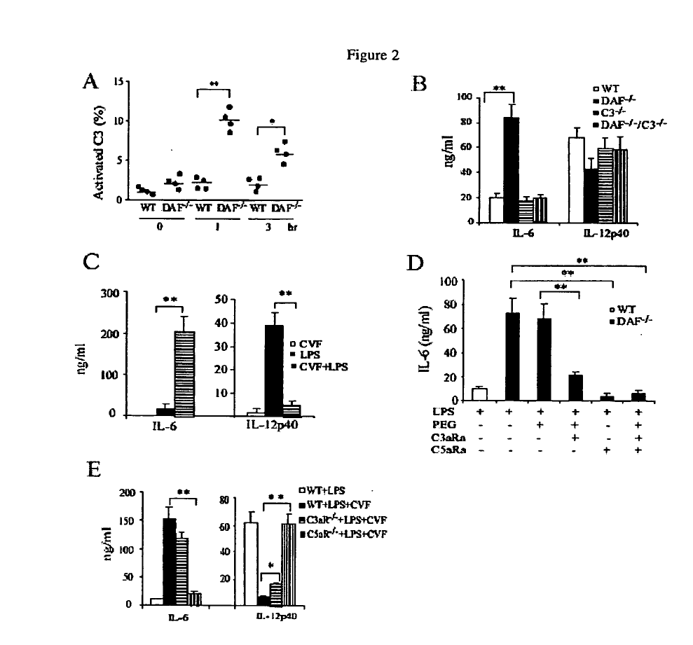

LPS is an activator of the alternative and lectin pathways of complement. Using activated plasma C3 fragments as a measure, a significantly (p<0.001) higher degree of complement activation in DAF^{-/-} micewas detected compared to the wild-type mice at 1 and 3 hours after LPS injection (FIG. 2A). This result suggested an important role of DAF in preventing LPS-induced complement activation in vivo. To test the hypothesis that changes in LPS-induced cytokine production in DAF^{-/-} mice were caused by increased complement activation, the LPS responses of DAF^{-/-}/C3^{-/-} mice were examined. As shown in FIG. 2B, increased plasma IL-6 and decreased IL-12p40 concentrations in DAF^{-/-} mice. However, similar changes in cytokine production were not observed in DAF^{-/-}/C3^{-/-} or C3^{-/-} mice (FIG. 2B). Thus, changes in LPS-induced cytokine production in DAF^{-/-} mice were completely dependent on complement. Furthermore, the phenotype of altered LPS-induced cytokine production in DAP^{-/-} mice was TLR4 dependent as DAF^{-/-}/TLR4^{-/-} mice, like TLR4^{-/-} mice, were non-responsive to LPS stimulation.

We next investigated if coincidental complement activation could also regulate TLR4 signaling in wild-type mice. CVF is a potent complement activator that, when given systemically, can overwhelm the complement regulatory mechanisms and cause extensive complement activation in normal animals. We treated wild-type mice with either LPS, CVF or the combination of the two. FIG. 2C shows that CVF treatment alone had negligible effect on IL-6 and IL-12p40 production. However, CVF co-treatment greatly increased LPS-induced plasma 1L-6 and decreased LPS-induced plasma IL-12p40 concentrations (FIG. 2C). This result supported the conclusion that increased complement activation, rather than DAF deficiency per se, caused the observed changes in LPS-induced cytokine production in DAF^{-/-} mice.

Complement activation generates multiple bioactive peptides including the anaphylatoxins C3a and C5a, as well as the membrane attack complex. To determine which downstream complement mediator(s) was responsible for interacting with the TLR4 pathway, we treated DAF^{-/-} mice with SB 290157, a C3a receptor (C3aR) antagonist³⁵, and AcPhe, a cyclic peptide C5a receptor (C5aR) antagonist, either alone or in combination. FIG. 2D shows that the increase in LPS-induced IL-6 production in DAF^{-/-} mice was significantly (p<0.001) attenuated by SB 290157 and totally blocked by the C5aR antagonist. In a parallel experiment, we investigated the role of C5aR and C3aR using mice deficient in C5aR or C3aR. FIG. 2E shows that C3aR deficiency partially corrected the abnormality in CVF-induced IL-6 and IL-12p40 production. Strikingly, C5aR deficiency almost completely reversed the CVF effect on IL-6 and IL-12p40 production (FIG. 2E). Thus, the regulatory effect of complement on TLR4 signaling in vivo appeared to be mediated by C5aR and, to a much lesser extent, C3aR signaling.

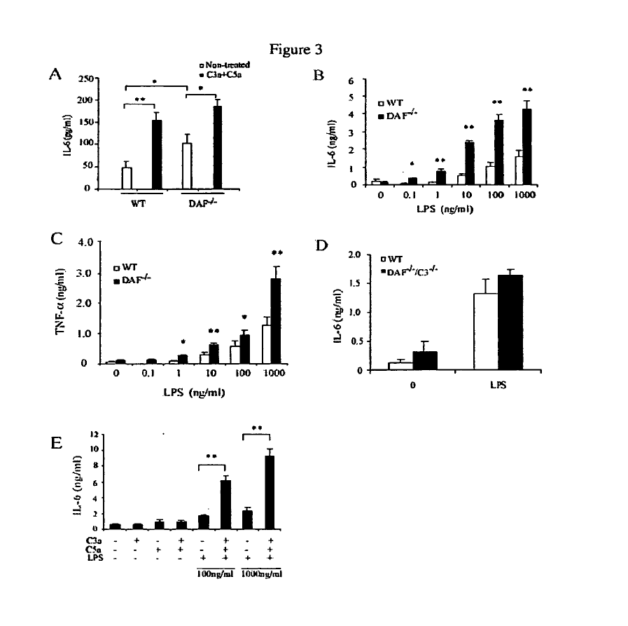

Next the effect of C5a and C3a on mouse splenocytes and thioglycolate-elicited peritoneal macrophages in vitro was examined. Both types of cells are known to express TLR4, C5aR and C3aR and this was confirmed by RT-PCR and/or FACS analysis. Splenocytes from LPS-challenged wild-type and DAF^{-/-} mice were isolated and cultured in the presence or absence of C5a/C3a. In the absence of C5a/C3a, cultured DAF^{-/-} splenocytes secreted higher amount of IL-6 than wild-type cells (FIG. 3A), presumably reflecting a carryover effect of complement activation on LPS signaling in vivo. Notably, supplementation of C5a/C3a to cells in culture significantly (p<0.05) augmented IL-6 production by both wild-type and DAF^{-/-} cells (FIG. 3A). We also found that cultured peritoneal macrophages from DAF^{-/-}, but not DAF^{-/-}/C3^{-/-}, mice produced higher amounts of IL-6 and TNF-α than wild-type macrophages in response to LPS stimulation (FIG. 3B-3D). As with splenocytes, addition of C5a/C3a to wild-type mouse peritoneal macrophages in culture augmented LPS-mediated IL-6 production (FIG. 3E).

### EXAMPLE 3

### Altered LPS-Induced Cytokine Production in DAF^{-/-} Mice Involved Increased NF-KB and Mapk Signalling

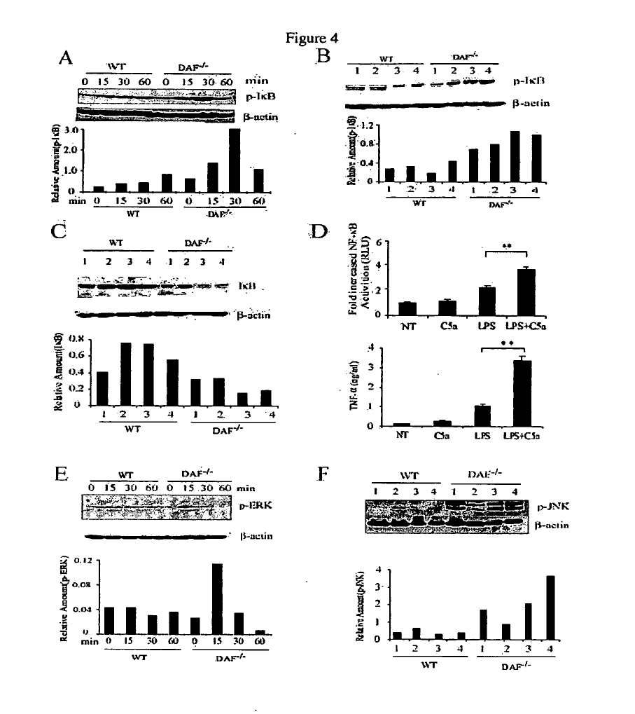

TLR4-induced inflammatory cytokine production involves NF-kB activation. It was found in the present set of experiments that LPS induced a more rapid and robust NF-kB activation in the spleens of . DAF^{-/-} mice than in wild-type mice (FIG. 4A-4C). Increased phosphorylation of the NF-kB inhibitor IkB-β was detected at 15 minutes and 30 minutes after LPS stimulation in the spleens of DAF^{-/-} mice (FIG. 4A, 4B). Correspondingly, we found that total IkB-β levels in the spleens of DAF^{-/-} mice were significantly decreased at 60 minutes after LPS stimulation (FIG. 4C). Thus, altered LPS-induced cytokine production in DAF^{-/-} mice was correlated with increased activation of the NF-kB pathway. To directly test the involvement of NF-κB, we transfeceted RAW264.7 cells with an NF-κB luciferase reporter gene and studied the possible synergistic activation of NF-kB by LPS and C5a. FIG. 4D shows that C5a had negligible effect on its own but it synergized with LPS in stimulating the expression of the NF-kB reporter gene as well as the secretion of endogenous TNF-α.

Both C5aR and C3aR belong to the G-protein coupled receptor (GPCR) superfamily of membrane proteins. One of the downstream intracellular signaling pathways of C5aR and C3aR ligation is the activation of MAP kinases by phosphorylation. TLR-induced intracellular signaling also involves MAP kinase activation. To determine the possible role of MAP kinases in the altered LPS-induced cytokine production in DAF^{-/-} mice, the activation kinetics of the extracellular signal regulated kinase (ERK1/2), the c-Jun amino terminal kinase (JNK) and p38 MAP kinases were compared in LPS-treated wild-type and DAF^{-/-} mice. No difference in the phosphorylation of p38 in the spleens of wild-type and DAF^{-/-} mice were detected. On the other hand, after LPS stimulation, significant increase in ERK1/2 and JNK phosphorylation was observed in the spleens of DAF^{-/-} mice (FIG. 4E, 4F).

### EXAMPLE 4

### Complement Also Regulates TLR2/6 and TLR9 Signalling

To determine if the regulatory effect of complement on TLR4 signaling is also observed with other TLRs, wild-type and DAF^{-/-} mice were treated with zymosan, a TLR2/TLR6 ligand and a well known activator of the alternative pathway complement. As in LPS-induced TLR4 signaling, it was found that zymosan-induced IL-6, TNF-α and IL-1β production was also significantly increased in DAF^{-/-} mice (FIG. 5A). In a parallel experiment, wild-type and MyD88^{-/-} mice were challenged with zymosan, either alone or in combination with CVF. Markedly increased IL-6 and decreased IL-12p40 production were detected in wild-type mice co-treated with zymosan and CVF (FIG. 5B). Importantly, it was found that IL-6 and IL-12 production was abrogated in MyD88^{-/-} mice treated with either zymosan or zymosan/CVF (FIG. 5B), suggesting that complement interacted with zymosan-triggered TLR2/6 signaling and not with the zymosan-mediated dectin pathway.

Next the responses of wild-type and DAF^{-/-} mice to CpG oligodeoxynucleotide (CpG ODN), a prototypical ligand for the intracellularly localized TLR9 were examined. No significant differences between the two groups of mice in their plasma IL-6, TNF-α or IL-1β concentration were detected (FIG. 5C). On the other hand, it was found that DAF^{-/-} mice produced significantly (p<0.05) less IL-12p40 than wild-type mice in response to CpG challenge (FIG. 5C). Surprisingly,-this phenotype of reduced IL-12 production was rescued in DAF^{-/-}/C3^{-/-} but not DAF^{-/-}/C5aR^{-/-} mice (FIG. 5C). These observations suggested: a) that CpG may activate complement in vivo and, b) that unlike in LPS-triggered TLR4 activation, effector(s) other than C5a may be principally responsible for the complement-dependent suppression of CpG-induced IL-12p40 production. Indeed, analysis of plasma samples of CpG-treated mice showed detectable complement activation and the degree of complement activation was higher in CpG-treated DAF^{-/-} mice than in similarly-treated wild-type mice. To corroborate the findings in DAF^{-/-} mice, we investigated the effect of CVF-induced complement activation on CpG-stimulated cytokine production in wild-type mice. Consistent with the result from DAF^{-/-} mice, it was found that CVF co-treatment had no significant impact on CpG-induced IL-6, TNF-α or IL-1β production but markedly suppressed IL-12p40 production (FIG. 5D). Notably, unlike the CVF effect on LPS-induced IL-12p40 production which was predominantly mediated by C5aR (FIG. 2E), the inhibitory effect of CVF treatment on CpG-induced IL-12p40 production was only moderately corrected by C5aR deficiency but was substantially reversed by C3aR deficiency (FIG. 5E).

### EXAMPLE 5

### Mechanism of Complement-Mediated IL-12 Suppression

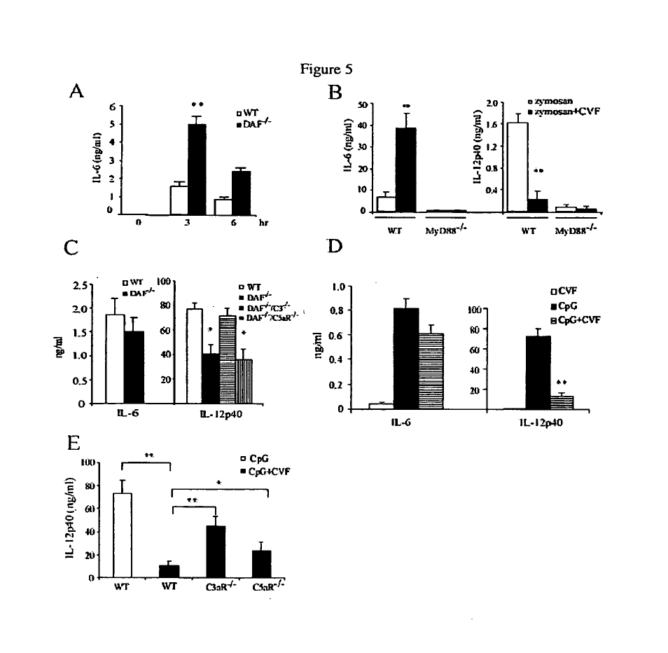

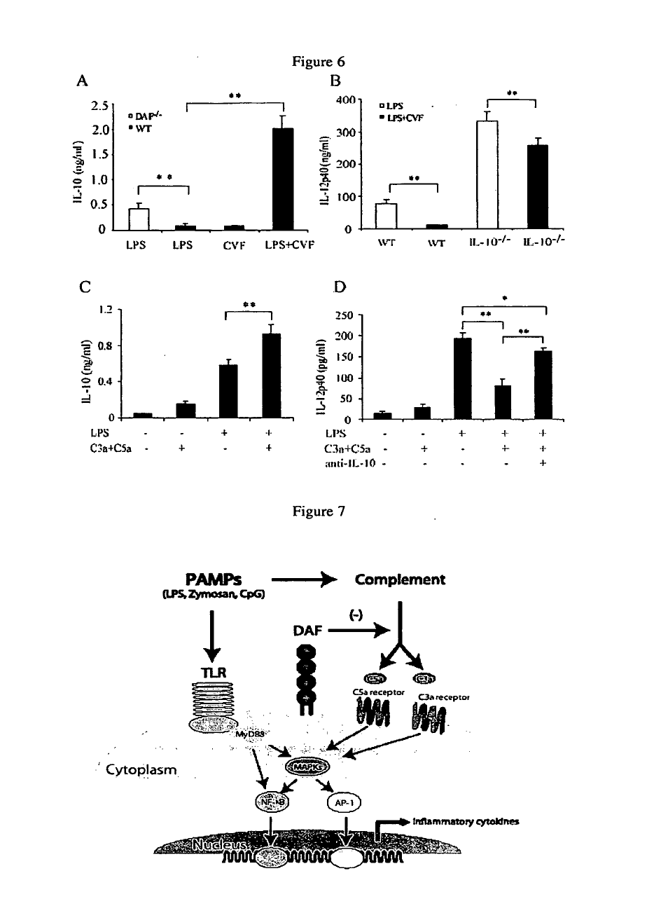

The suppression by complement of LPS-induced IL-12 production contrasted with its strong stimulating effect on IL-6, TNF-α and IL-1β. To investigate this paradoxical phenomenon, we examined the production of IL-10, an inhibitory cytokine that is known to regulate IL-12 biosynthesis, in wild-type and DAF^{-/-} mice challenged with LPS or LPS/CVF. FIG. 6A shows that IL-10 level was significantly higher in LPS-treated DAF^{-/-} mice and strikingly elevated in LPS/CVF-treated wild-type mice as compared with LPS- or CVF-treated wild-type mice. To test if IL-10 regulated IL-12 production under our experimental setting, we measured IL-12p40 production in IL-10^{-/-} mice after LPS or LPS/CVF challenge. FIG. 6B shows that compared with wild-type mice, IL-10^{-/-} mice produced much higher levels of IL-12p40 in response to LPS or LPS/CVF stimulation, confirming that IL-10 is a negative regulator of IL-12 production in vivo. Of interest, we found that, as in wild-type mice, CVF co-treatment suppressed LPS-induced IL-12p40 production in IL-10^{-/-} mice, suggesting an IL-10-independent effect of complement on IL-12p40 production. It is notable, however, that the magnitude of IL-12p40 suppression by CVF treatment was considerably reduced in IL-10^{-/-} mice as compared with that in wild-type mice (22% vs 87% reduction). These results suggested that complement may have inhibited IL-12 production in vivo through both IL-10-dependent and -independent mechanisms.

To further examine the intermediacy of IL-10 in complement-mediated IL-12 suppression, we measured IL-10 production by cultured peritoneal macrophages. We found that C5a and C3a significantly increased LPS-stimulated IL-10 (FIG. 6C) and decreased LPS-stimulated IL-12p40 (FIG. 6D) Production in cultured peritoneal macrophages. Importantly, addition to the cell culture medium of an IL-10 neutralization mAb largely reversed the suppressive effect of C5a/C3a on IL-12p40 production by these cells (FIG. 6D).

Despite many parallels between the TLR and the complement pathways, very little is known about their potential interactions in vivo. In this study, we have provided evidence for a strong interaction between complement and TLR signaling.

The data presented demonstrates that TLR4 -induced production of IL-6, IL-10, TNF-α and IL-1β was markedly increased, whereas that of IL-12 was decreased, in DAF^{-/-} mice. The complement-dependent nature of the DAF^{-/-} mouse phenotype in response to LPS challenge suggested that the phenomenon was related to DAF as a complement regulator rather than a LPS co-receptor. This conclusion is supported by the findings that plasma LPS concentrations were similar in DAF^{-/-} and wild-type mice, and that CVF-induced complement activation had similar effect on cytokine production in wild-type mice. Furthermore, the phenotype of DAF^{-/-} mice was not limited to LPS challenge but was observed also when these mice were challenged with zymosan or CpG oligonucleotide, respective ligand of TLR2/6 and TLR9. Whether the activity of DAF in preventing LPS- and other TLR ligand-induced complement activation in vivo is unique or shared by other complement regulators such as Crry, membrane cofactor protein (MCP) or factor H remains to be determined.

It was notable that the regulatory effect of complement on TLR4 -mediated cytokine production was correlated with the degree of complement activation. At the sub-lethal LPS dosage used, we detected no difference in cytokine production between wild-type and C3^{-/-} mice, suggesting that in the presence of DAF, limited LPS-triggered complement activation did not affect TLR4 signaling. Increased complement activation in DAF^{-/-} mice significantly augmented LPS-dependent IL-6, TNF-α, IL-1β and IL-10 production but only moderately inhibited IL-12 production. In contrast, CVF-induced overwhelming complement activation markedly increased IL-6 and IL-10 and dramatically decreased IL-12p40 production in wild-type mice. Our finding of IL-12 inhibition by complement in vivo is consistent with the report of Hawlisch et al who demonstrated a similar phenomenon in cultured murine peritoneal macrophages. Our data suggested that the inhibition of IL-12 production by complement involved both IL-10-dependent and -independent mechanisms.

Through the use of receptor antagonists and C3aR^{-/-} and C5aR^{-/-} mice, it was shown that the regulatory effect of complement on TLR signaling was mediated by C5a and C3a. It was notable that the effect on TLR4 signaling by complement was predominantly mediated by C5aR, whereas C3aR played a more important role than C5aR in regulating TLR9 signaling. This difference may have reflected differential interaction of C5aR/C3aR signaling with the TLR4 and TLR9 pathways, or a difference in C5aR and C3aR expression levels on cells responding to TLR4 and TLR9 ligation. The target cells of C5a and C3a action in vivo that contributed to the observed changes in plasma cytokine concentrations are yet to be fully characterized. Northern blot analysis showed increased IL-6 mRNA levels in several tissues of LPS-treated DAF^{-/-} mice including the spleen, lung and fat, suggesting that tissue macrophages and/or endothelial cells may be among the responding cells.

A quicker and more robust NF-kB activation was detected in the spleens of LPS-treated DAF^{-/-} mice, and demonstrated a synergistic effect of C5a on LPS-induced NF-kB reporter gene induction in RAW267.4 cells. These findings suggested that C5a/C3a-generated signals interacted with the TLR4 pathway upstream of the NF-kB activation step and amplified the normal TLR4 -dependent signal transduction (FIG. 7). Notably, we observed increased phosphorylation of the MAP kinases ERK1/2 and INK in LPS-challenged DAF^{-/-} mouse spleens. These results collectively suggested that MAPKs may be the key molecules linking the two pathways together (FIG. 7). A further potential interaction between the TLR and complement, not mutually exclusive with the sequence of events depicted in FIG. 7, was that TLR-induced inflammatory cytokines up-regulated the expression of C5aR and C3aR.

It was unexpected that DAF, a cell membrane protein, effectively regulated LPS-induced systemic complement activation in vivo which has been thought to occur largely in the fluid phase. LPS may have incorporated into or associated with the cell membrane through micelle formation or binding to membrane proteins (e.g. CD14, TLR4). Thus, LPS-induced complement activation may have occurred on or near the cell surface where it was subjected to regulation by DAF. This scenario is compatible with the observed increase in IL-6 and TNF-α production by LPS-stimulated DAF^{-/-} macrophages in culture (FIG. 3). Macrophages are a well-known source of extrahepatic complement proteins and were presumably self-sufficient in supporting LPS-induced C5a/C3a generation in the absence of DAF. In support of this hypothesis, it was found that C3 deficiency rescued the phenotype of DAF^{-/-} macrophages, i.e no difference in IL-6 and TNF-α production was observed between LPS-stimulated DAF^{-/-}/C3^{-/-} and wild-type macrophages in culture (FIG. 3).

Thus, this data revealed a widespread and striking regulatory effect of complement on TLR signaling in vivo. these findings suggest a novel mechanism by which complement promotes inflammation and modulates adaptive immunity and provide new insight into the interaction between two essential innate immune systems relevant to host-pathogen interaction, autoimmunity and vaccine development.

### EXAMPLE 6

### Complement Activation Products, Particularly C5A, Synergize with the TLR Activation Pathway to Drive TH-17 T Cell Differentiation

Th-17 cells, characterized by IL-17 production, are critical in many autoimmune diseases. Inflammatory cytokines, particularly IL-6, TNF-α and IL-1b, in conjunction with TGF-β are critical for Th-17 cell differentiation.

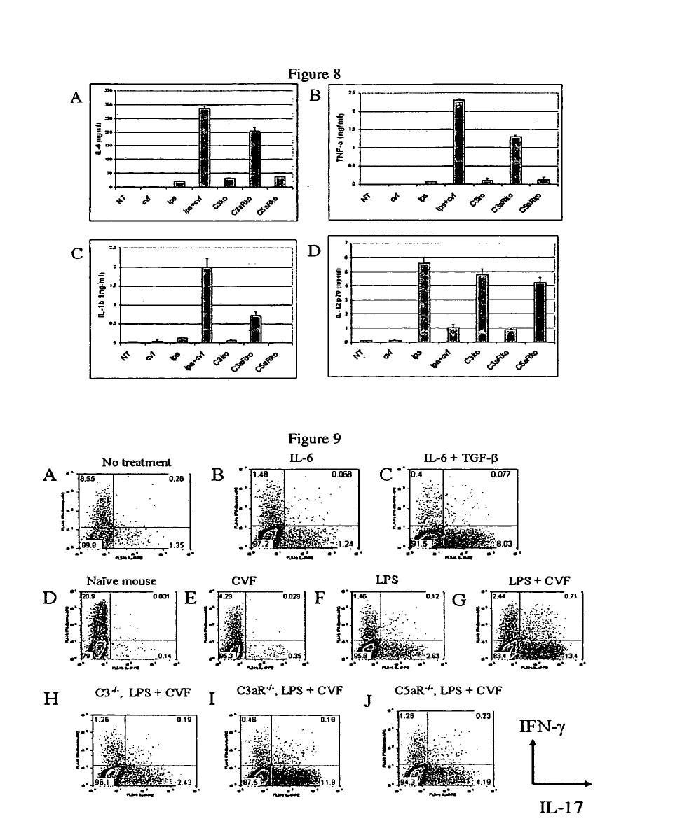

A set of experiment was conducted to measure serum concentrations of cytokines in mice untreated (NT) or treated with TLR and/or complement activators. The results obtained show that the complement activator CVF synergized with the TLR4 ligand LPS to produce augmented IL-6, TNF-α, and IL-1β. On the other hand, CVF reduced LPS-induced IL-12 production (FIG. 8). Thus, the effect of CVF on LPS-induced cytokine production is dependent on complement C3 and C5aR but not C3aR signaling.

To test whether the CVF-augmented inflammatory cytokine production promotes Th-17 differentiation, purified naive CD4 T cells from wild-type mice were stimulated in vitro with plate-bound anti-CD3 and CD28 in the presence of specific cytokines or sera of control (naive mouse) or LPS-, CVF-treated mice. CD4 T cells were differentiated into Th-17 cells by IL-6 in the presence of TGF-β. Untreated (naive) or CVF-treated mouse sera were unable to drive Th-17 differentiation. Serum from LPS-treated mice was active in driving Th-17 differentiation and this effect was strongly augmented by CVF co-treatment (FIG. 9). The augmenting effect of CVF on LPS-dependent Th-17 differentiation required C3 and C5aR but not C3aR. These data are in agreement with the inflammatory cytokine data presented in (FIG. 8).

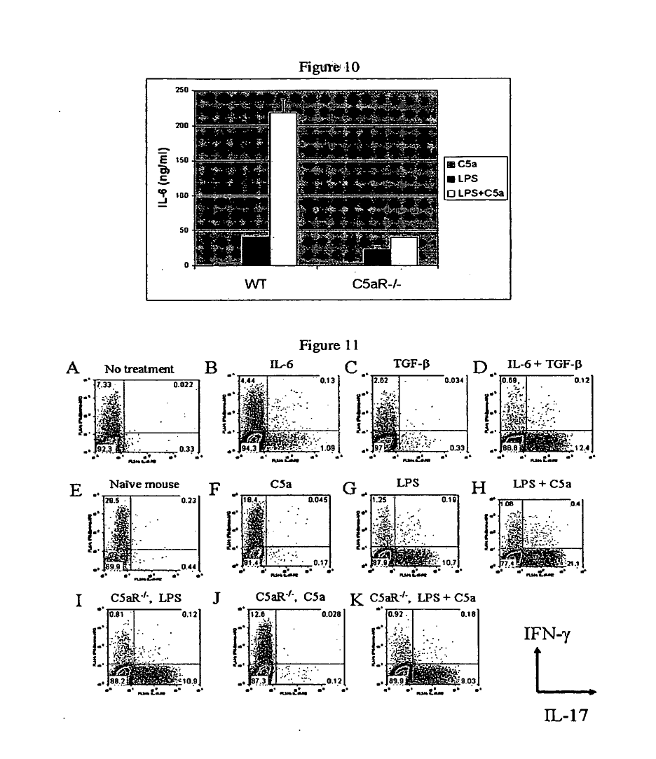

Next, the Synergistic effect of C5a with LPS in augmenting IL-6 production was tested. Instead of using the complement activator CVF, the complement activation product C5a was directly tested for its activity to synergize with LPS in IL-6 production. Mice treated with LPS and CVF had greatly elevated serum IL-6 level compared with mice treated with LPS alone. CVF treatment by itself has no effect on IL-6 production. Thus, the synergistic activity of C5a required C5aR as such an effect was not observed in C5aR^{-/-} mice (FIG. 10).

The ability of C5a-induced augmentation of serum inflammatory cytokine production to promote Th-17 differentiation was assessed. Purified naive CD4 T cells from wild-type mice were stimulated in vitro with plate-bound anti-CD3 and CD28 in the presence of specific cytokines or sera of control (naive mouse) or LPS-, C5a-treated mice. CD4 T cells were differentiated into Th-17 cells by IL-6 in the presence of TGF-β. Treatment of the cells with IL-6 alone or TGF-β alone had no effect on Th-17 differentiation. Untreated (naive mouse) or C5a-treated mouse sera were unable to drive Th-17 differentiation. Serum from LPS-treated mice was active in driving Th-17 differentiation and this effect was strongly augmented by C5a co-treatment (FIG. 11). Thus the augmenting effect of C5a on LPS-dependent Th-17 differentiation required C5aR. These data are in agreement with the IL-6 data presented in FIG. 3. These findings indicate that complement activation products, particularly C5a, synergize with the TLR activation pathway to augment inflammatory cytokine production. The complement system, through interaction with TLR, augments Th-17 differentiation and therefore plays a role in autoimmune tissue injury. FIGS. 8-11 provide evidence for this conclusion. Thus, inhibiting complement activation is a therapeutic approach for treating Th-17 T cell mediated autoimmune diseases such as multiple sclerosis, Lupus, inflammatory bowel disease, GvHD and transplant rejection.

What is claimed is:

1. A method of inducing the production of a pro-inflammatory cytokine selected from: an IL-6, an IL-10, a TNFα, an IL-1β, or any combination thereof in a subject, comprising the step of activating an anaphylatoxin receptor in said subject, thereby inducing the production of a pro-inflammatory cytokine in a subject.

2. The method of claim wherein said anaphylatoxin receptor is a C3a receptor (C3aR) or a C5a receptor (C5aR).

3. The method of claim 1, wherein the step of activating an anaphylatoxin receptor in said subject comprises activating the complement system in said subject.

4. The method of claim 4, wherein said activating the complement system in said subject comprises administering to said subject an inducer of the complement system.

5. The method of claim 5, wherein said inducer of said complement system is cobra venom factor (CVF).

6. The method of claim 4, wherein said activating the complement system in said subject comprises administering to said subject an inhibitor of complement degradation.

7. The method of claim 7, wherein said inhibitor of complement degradation is complement inhibitor decay-accelerating factor (DAF).

8. The method of claim 1, wherein said activating an anaphylatoxin receptor in said subject comprises administering to said subject a composition comprising a C3a protein, a C5a protein, or a combination thereof.

9. The method of claim 1, wherein said activating an anaphylatoxin receptor in said subject comprises administering to said subject a composition comprising an agonist of a C3a protein, a C5a protein, or a combination thereof.

10. The method of claim 1, wherein said inducing the production of a pro-inflammatory cytokine in said subject comprises activating both a Toll-like receptor (TLR) and said complement system in said subject.

11. The method of claim 11, wherein said activating both said TLR and said complement system in said subject comprises administering to said subject a composition comprising a lipopolysaccharide (LPS), or zymosan, or a combination thereof.

12. A method of preventing a Toll-like receptor (TLR) dependent inflammation in a subject, comprising the step of inhibiting an anaphylatoxin receptor in said subject, thereby preventing a Toll-like receptor (TLR) dependent inflammation in a subject.

13. The method of claim 12, wherein said preventing a TLR dependent inflammation in said subject comprises inhibiting the production of a pro-inflammatory cytokine selected from: IL-6, IL-10, TNFα, IL-1β, or any combination thereof in said subject.

14. The method of claim 12, wherein said anaphylatoxin receptor is a C3a receptor (C3aR) or a C5a receptor (C5aR).

15. The method of claim 12, wherein said TLR is a TLR2, a TLR4, a TLR6, or a TLR9.

16. The method of claim 12, wherein said TLR is expressed by an antigen-presenting cell.

17. The method of claim 12, wherein the step of inhibiting an anaphylatoxin receptor in said subject comprises inhibition of the complement system in said subject.

18. The method of claim 12, wherein the step of inhibiting an anaphylatoxin receptor in said subject comprises administering to said subject a C3aR antagonist, a C5aR antagonist, or a combination thereof.

19. The method of claim 18, wherein said C3aR antagonist is SB 290157.

20. The method of claim 18, wherein said C5aR antagonist is AcPhe.

21. A method of inducing an immune response against an antigen in a subject, comprising the step of activating an anaphylatoxin receptor in said subject, thereby inducing an immune response against an antigen in a subject.

22. The method of claim 21, wherein said inducing an immune response against an antigen in a subject comprises the production of a pro-inflammatory cytokine in said subject.

23. The method of claim 22, wherein said pro-inflammatory cytokine comprises IL-6, IL-10 TNFα, IL-1β, or any combination thereof.

24. The method of claim 21, wherein said anaphylatoxin receptor is a C3a receptor (C3aR)or a C5a receptor (C5aR).

25. The method of claim 21, wherein the step of activating an anaphylatoxin receptor in said subject comprises activating the complement system in said subject.

26. The method of claim 25, wherein said activating the complement system in said subject comprises administering to said subject an inducer of said complement system.

27. The method of claim 26, wherein said inducer of said complement system is cobra venom factor (CVF).

28. The method of claim 21, wherein said activating said complement system in said subject comprises administering to said subject an inhibitor of complement degradation.

29. The method of claim 28, wherein said inhibitor of complement degradation is complement inhibitor decay-accelerating factor (DAF).

30. The method of claim 21, wherein said activating an anaphylatoxin receptor in said subject comprises administering to said subject a composition comprising a C3a protein, a C5a protein, or a combination thereof.

31. The method of claim 21, wherein said activating an anaphylatoxin receptor in said subject comprises administering to said subject a composition comprising an agonist of C3a protein, a C5a protein, or a combination thereof.

32. The method of claim 21, wherein said inducing an immune response in said subject comprises activating both a Toll-like receptor (TLR) and said complement system in said subject.

33. The method of claim 32, wherein said activating both a TLR and said complement system in said subject comprises administering to said subject a composition comprising a lipopolysaccharide (LPS), or zymosan, or a combination thereof.

34. A method of inhibiting an immune response against an antigen in a subject, comprising the step of inhibiting an anaphylatoxin receptor in said subject, thereby inhibiting an immune response against an antigen in a subject.

35. The method of claim 34, wherein said inhibiting an immune response against an antigen in said subject comprises inhibiting the production of a pro-inflammatory cytokine selected from: IL-6, IL-10, TNFα, IL-1β, or any combination thereof in said subject.

36. The method of claim 34, wherein said anaphylatoxin receptor is a C3a receptor (C3aR) or a C5a receptor (C5aR).

37. The method of claim 34, wherein the step of inhibiting an anaphylatoxin receptor in said subject comprises inhibition of the complement system in said subject.

38. The method of claim 34, wherein the step of inhibiting an anaphylatoxin receptor in said subject comprises administering to said subject a C3aR antagonist, a C5aR antagonist, or a combination thereof.

39. The method of claim 38, wherein said C3aR antagonist is SB 290157.

40. The method of claim 38, wherein said C5aR antagonist is AcPhe.

41. A method of treating a Th 17 cell mediated disease in a subject comprising the step of inhibiting complement system activation in said subject, thereby treating a Th17 cell mediated disease in a subject.

42. The method of claim 41, wherein said Th17 cell mediated disease is an autoimmune disease.

43. The method of claim 42, wherein said autoimmune disease is multiple sclerosis, lupus, inflammatory bowel disease, graft versus host disease, or transplant rejection.

44. The method of claim 41, wherein said inhibiting complement system activation comprises administering to said subject a compstatin, an anti-CS monoclonal antibody, an anti-factor B monoclonal antibody, an anti-properdin monoclonal antibodies, a recombinant extracellular domain of CRIg, a recombinant DAF, a recombinant MCP, a recombinant DAF-MCP chimera protein, or any combination thereof.

45. The method of claim 41, wherein said inhibiting complement system activation comprises administering to said subject a C5a antagonist.

46. The method of claim 45, wherein said C5a antagonist is C5aRA A8^{Δ71-73}.

47. A vaccine comprising an antigen, a Toll-like receptor (TLR) ligand, and an inducer of said complement system.

48. The vaccine of claim 47, wherein said antigen is a cancer antigen, bacterial antigen, or a viral antigen.

49. The vaccine of claim 47, wherein said TLR is a TLR2, a TLR4, a TLR6, or a TLR9.

50. The vaccine of claim 47, wherein said TLR ligand and said inducer of said complement system is a lipopolysaccharide (LPS), or zymosan.

51. The vaccine of claim 41, wherein said inducer of said complement system is cobra venom factor (CVF).

52. The vaccine of claim 41, wherein said inducer of said complement system is a C3a protein, a C5a protein or a combination thereof.

53. A vaccine comprising an antigen, a Toll-like receptor (TLR) ligand, and an inhibitor of complement degradation.

54. The vaccine of claim 53, wherein said antigen is a cancer antigen, bacterial antigen, or a viral antigen.

55. The vaccine of claim 53, wherein said TLR is a TLR2, a TLR4, a TLR6, a TLR9, or any combination thereof.

56. The vaccine of claim 53, wherein said Toll-like receptor (TLR) ligand is a lipopolysaccharide (LPS), or zymosan.

57. The vaccine of claim 53, wherein said inhibitor of complement degradation is complement inhibitor decay-accelerating factor (DAF).
